(19) United States
(12) Patent Application Publication
(10) Pub. No.: US 2024/0249844 A1
(43) Pub. Date: Jul. 25, 2024

(54) DIAGNOSTIC IMAGING DEEP LEARNING SYSTEM AND METHOD

(71) Applicant: IMERA.AI Limited, London (GB)

(72) Inventors: Martin Siebachmeyer, London (GB); Jalaj Jain, London (GB); Krishna Boddu, Ipswich (GB); Sandeshkumar Gangadharappa Lakkol, Derby (GB)

(21) Appl. No.: 18/624,949

(22) Filed: Apr. 2, 2024

Related U.S. Application Data
(63) Continuation-in-part of application No. PCT/IB2022/059146, filed on Sep. 26, 2022.
(60) Provisional application No. 63/254,480, filed on Oct. 11, 2021.

(51) Int. Cl. G16H 50/20; G06T 7/11; G06V 10/25; G06V 10/764; G06V 20/70; G16H 30/40

(52) U.S. Cl. CPC G16H 50/20; G06T 7/11; G06V 10/25; G06V 10/764; G06V 20/70; G16H 30/40; G06T 2207/20132

(57) ABSTRACT

A method includes receiving, by at least one processor, a diagnostic image, detecting, by the at least one processor using a pathology detection network, a primary pathology of the diagnostic image, locating, by the at least one processor using an anatomy network, at least one anatomical region of the diagnostic image, classifying, by the at least one processor using an attribute classification network, one or more attributes of the primary pathology, and generating, by the at least one processor, an output based on the primary pathology, the at least one anatomical region, and the one or more attributes.

### BACKGROUND

Diagnostic images are generated in a very large volume and their analysis represents a large and time consuming portion of some medical professionals' careers. It can be difficult to review and analyze these images and provide feedback in a timely fashion with a high accuracy.

There are many existing approaches to overcome these challenges. However, each of the approaches has its own limitations. A first approach is based on image level classification. In this approach, a full X-ray image is classified to binary or multiclass labels. An image saliency map is produced to visualize the decision making of the deep learning model.

A second approach is a semi-automated approach that includes fracture detection in the specific anatomical region. In this approach, a specific anatomical region (region-of-interest (ROI)), is manually extracted from a full X-ray image. Then, a ROI is classified to one of the following: (1) Binary classification: Normal vs. Abnormal anatomical region and (2) Multi class classification: More than two classification categories.

A third approach is a fully automated approach for fracture detection in the specific anatomical region. In this approach, a specific anatomical region (region-of-interest (ROI)) is automatically classified from a full X-ray image, using two networks. A first network is responsible for automatic localization and classification of a region-of-interest (ROI) anatomical region. The ROI is fed to a second network as an input. The second network is responsible for classification of region-of-interest (ROI) anatomical region (output of the first network).

However, there are many challenges with these approaches. The first approach is limited to solved binary classification problems such as a Normal vs. Abnormal image. In addition, it produces a saliency-map that highlights the model decision making process. While being attractive, this approach can also lead to wrong interpretations as there is no guarantee that the deep learning network is highly accurate to identify the casual relationships between image features and output. Often in the presence of multiple abnormalities, the saliency map is inaccurate.

The second and third approach also have numerous issues. They are practically not scalable. As an example, a pelvic-AP X-ray image has the following twenty-six anatomical regions:

```
  Hip ( femoral-head, femoral-neck, intertroch-femur, subtroch-femur,
greater-troch, lesser-troch, prox-femur, femoral-shaft)
  Ilium
  Spine (L1_body, L2_body,L3_body, L4_body, L1_transverse,
L2_transverse, L3_transverse, L4_transverse, L5_transverse)
  ASIS, AIIS, sacral_ala, sacral_body, cocyx and ischium
  Acetabulum
```

With this approach, the twenty-six anatomical regions are to be cropped and fed to the classification network in the pelvic-AP alone. To achieve a clinically relevant performance, a large amount of the training datasets is needed since each anatomical region is classified separately. There is no formal learning for the deep learning network to understand the features of fractures in the different anatomical regions.

It is with these issues in mind, among others, that various aspects of the disclosure were conceived.

### SUMMARY

According to one aspect, a diagnostic imaging deep learning system and method is provided that finds, locates, and classifies one or more pathologies in one or more diagnostic images using deep learning networks. In one example, the system may find and locate the one or more pathologies or abnormalities using multiple deep learning networks in a hierarchical manner. The system may use an adaptive runtime image processing algorithm to retain high resolution image information during deep learning network training and inference. The system may combine output from multiple deep learning networks. Each network may locate and classify specific pathology class labels. The system may create and store pathology signatures/descriptors and compare the signatures/descriptors to signatures/descriptors computed from pathologies/abnormalities in a test image. In addition, the system may provide end-to-end processing to (1) generate a diagnostic report such as an X-ray diagnostic report, and/or (2) highlight an area of concern to provide information for clinical staff, and/or (3) prioritize abnormal images with abnormality finding. In addition, the system may manage risk by tagging one or more output bounding boxes to have a confidence label such as a high confidence label or a low confidence label.

In one example, a method may include receiving, by at least one processor, a diagnostic image, detecting, by the at least one processor using a pathology detection network, a primary pathology of the diagnostic image, locating, by the at least one processor using an anatomy network, at least one anatomical region of the diagnostic image, classifying, by the at least one processor using an attribute classification network, one or more attributes of the primary pathology, and generating, by the at least one processor, an output based on the primary pathology, the at least one anatomical region, and the one or more attributes.

In another example, a system may include at least one processor and a memory having instructions stored thereon and executed by the at least one processor to: receive a diagnostic image, detect using a pathology detection network, a primary pathology of the diagnostic image, locate using an anatomy network, at least one anatomical region of the diagnostic image, classify using an attribute classification network, one or more attributes of the primary pathology, and generate an output based on the primary pathology, the at least one anatomical region, and the one or more attributes.

In another example, a non-transitory computer-readable storage medium includes instructions stored thereon that, when executed by at least one computing device cause the at least one computing device to perform operations, the operations including receiving a diagnostic image, detecting using a pathology detection network, a primary pathology of the diagnostic image, locating using an anatomy network, at least one anatomical region of the diagnostic image, classifying using an attribute classification network, one or more attributes of the primary pathology, and generating an output based on the primary pathology, the at least one anatomical region, and the one or more attributes.

These and other aspects, features, and benefits of the present disclosure will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a block diagram of a diagnostic imaging deep learning system according to an example of the instant disclosure.

FIGS. 2-5 show diagnostic images processed by the system according to an example of the instant disclosure.

FIG. 6 shows an image divided into regions according to an example of the instant disclosure.

FIG. 7 shows a flow diagram of the diagnostic imaging deep learning system according to an example of the instant disclosure.

FIGS. 8-10 show an image divided into tiles by the system according to an example of the instant disclosure.

FIG. 11 shows a flow diagram associated with training according to an example of the instant disclosure.

FIGS. 12-16 show flow diagrams associated processing a diagnostic image according to an example of the instant disclosure.

FIG. 17 shows an example of a processed diagnostic image according to an example of the instant disclosure.

FIGS. 18-19 show model uncertainty computation according to an example of the instant disclosure.

FIG. 20 shows a flow diagram associated with an image pipeline according to an example of the instant disclosure.

FIG. 21 is a flowchart of a method for processing a diagnostic image according to an example of the instant disclosure.

FIGS. 22 and 23 are images showing an example of showing high and low confidence output on an output x-ray image.

FIG. 24 shows how methods of the present invention can learn the main class and its sub-classification of an x-ray image.

FIG. 25 shows how methods of the present invention use the attribute classification network for sub-classification using multi-label classification, in parallel.

FIG. 26 shows how methods of present invention include a learning process that jointly learns the fracture main classification label and its subclassification label.

FIG. 27 illustrates an end-to-end block diagram to generate a diagnostic report.

FIG. 28 shows the two templates, one for presence of fracture and second for absence of fracture and an example illustrating the presence and absence of fracture.

FIG. 29 shows the iMERA AI detection results and corresponding example diagnostic report.

FIGS. 30 through 37 illustrate a segmentation guided process for analyzing x-ray images, according to exemplary embodiments of the present invention.

FIGS. 38 through 42 illustrate methods of eliminating the false bounding boxes, according to exemplary embodiments of the present invention.

FIGS. 43 and 44 illustrate methods of semi-automated online learning, according to exemplary embodiments of the present invention.

FIGS. 45 through 47 illustrate methods of building a training dataset for classification and detection of x-ray images, according to exemplary embodiments of the present invention.

FIG. 48 shows an example of a system for implementing certain aspects of the present technology.

### DETAILED DESCRIPTION

Aspects of a system and method for processing a diagnostic image includes finding, locating, and classifying one or more pathologies in one or more diagnostic images using deep learning networks. In one example, the system may find and locate the one or more pathologies or abnormalities using multiple deep learning networks in a hierarchical manner. The system may use an adaptive runtime image processing algorithm to retain high resolution image information during deep learning network training and inference. The system may combine output from multiple deep learning networks. Each network may locate and classify specific pathology class labels. The system may create and store pathology signatures/descriptors and compare the signatures/descriptors to signatures/descriptors computed from pathologies/abnormalities in a test image. In addition, the system may provide end-to-end processing to (1) generate a diagnostic report such as an X-ray diagnostic report, and/or (2) highlight an arca of concern to provide information for clinical staff, and/or (3) prioritize abnormal images with abnormality finding. In addition, the system may manage risk by tagging one or more output bounding boxes to have a confidence label such as a high confidence label or a low confidence label.

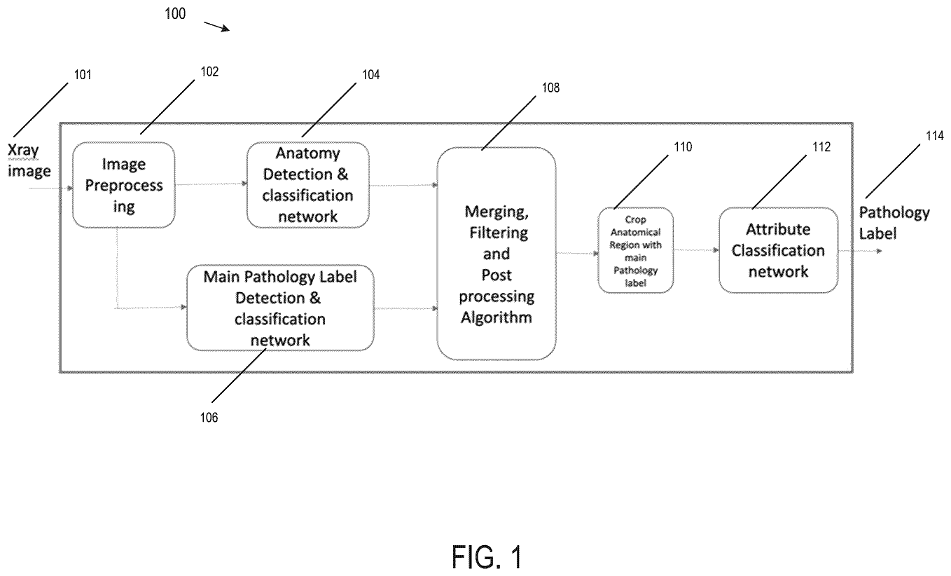

FIG. 1 illustrates a block diagram of a diagnostic imaging deep learning system 100 according to an example of the instant disclosure. As shown in FIG. 1, a diagnostic image 101 may be received and may be pre-processed 102. The system may use one network to perform anatomy detection and classification 104 and may use another network to perform main pathology label detection and classification 106. Next, the system may merge, filter, and perform post processing on the image 108. The system may select a particular subsection or region of the image by cropping an anatomical region having a main pathology label 110. The system may use a network to perform attribute classification 112 and may output an image having one or more pathology labels 114.

The system 100 may include at least one computing device that may be configured to receive data from and/or transmit data through a communication network. Although each computing device can be a single computing device, it is contemplated each computing device may include multiple computing devices.

The communication network can be the Internet, an intranet, or another wired or wireless communication network. For example, the communication network may include a Mobile Communications (GSM) network, a code division multiple access (CDMA) network, 3rd Generation Partnership Project (GPP) network, an Internet Protocol (IP) network, a wireless application protocol (WAP) network, a WiFi network, a Bluetooth network, a satellite communications network, or an IEEE 802.11 standards network, as well as various communications thereof. Other conventional and/or later developed wired and wireless networks may also be used.

Each computing device may include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions. In addition, each computing device further includes at least one communications interface to transmit and receive communications, messages, and/or signals.

Each computing device could be a programmable logic controller, a programmable controller, a laptop computer, a smartphone, a personal digital assistant, a tablet computer, a standard personal computer, or another processing device. Each computing device may include a display, such as a computer monitor, for displaying data and/or graphical user interfaces. The system may also include a Global Positioning System (GPS) hardware device for determining a particular location, an input device, such as a camera, a keyboard or a pointing device (e.g., a mouse, trackball, pen, or touch screen) to enter data into or interact with graphical and/or other types of user interfaces. In an exemplary embodiment, the display and the input device may be incorporated together as a touch screen of the smartphone or tablet computer.

The system 100 may utilize an annotated dataset that may provide a complete representation of X-ray pathology or abnormalities. The annotated dataset may include a plurality of images that have hierarchical labeling. The hierarchical labeling depth may depend on a type of pathology. For example, a, hierarchical representation of a pathology ‘fracture’ in pelvis Xray images, can be represented as below.

```
“Fracture-Femoral_neck- Displaced- callus_sclerotic_edges-Left″
  Where:
    ‘Fracture’: Pathology class label or type
    ‘Femoral_neck’: Anatomical region (Pelvic-AP bone)
    ‘Displaced’: Main attribute of pathology label
    ‘callus_sclerotic_edges'- Sub Attribute-1 of pathology label
    ‘Left’: Location of Pelvis anatomical region with respect to center
  (Left or Right or nil).
```

For hierarchical classification and localization of pathologies, an image may be processed using one or more networks including an anatomy network that may locate and classify one or more anatomical regions in the image, a pathology detection network that detects and classifies a main pathology label, and an attribute classification network to classify sub-attributes of a pathology class label. The attribute classification network may be used to multi-task and provide a multi-labeling network. Output from the one or more networks may be processed by a post processing algorithm. The post processing algorithm may determine an intersection of union (IoU) of bounding boxes of the networks and measure the IoU to determine one or more anatomical regions having a pathology. In addition, the post processing algorithm may perform false pathology detection to determine a pathology detection bounding box that may not overlap with an anatomical region bounding box. After post processing for each anatomic region having a pathology, the system may select a particular region in the image and crop the region and feed the region to an attribute classifier network. The attribute classifier network provides one or more labels and may be a multi-tasking classifier by providing one or more sub-attributes.

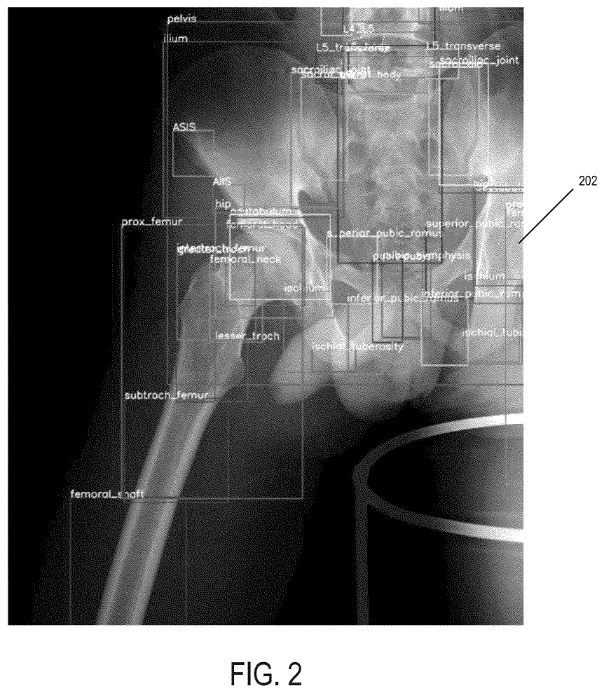

FIGS. 2-5 show example diagnostic images processed by the system according to an example of the instant disclosure. As shown in FIG. 2, there are one or more selected regions 202 found in FIG. 2. A first object detection network may locate and classify anatomical regions of the pelvic-anteroposterior (AP) image. As shown in FIG. 2, the first deep learning object detection network may classify and locate anatomical regions in the pelvic-AP image.

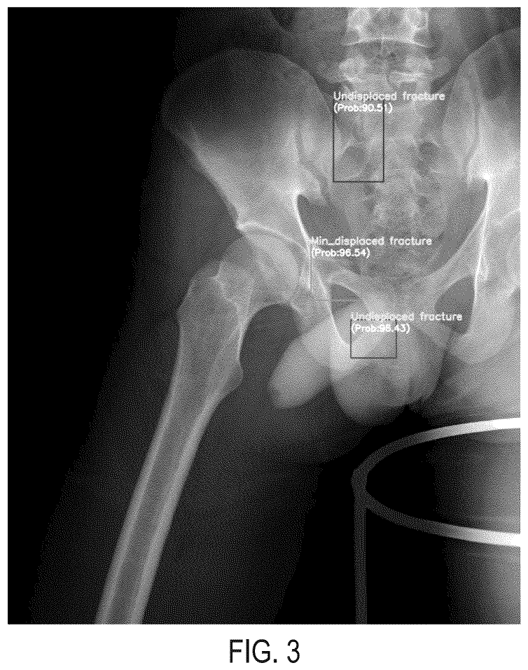

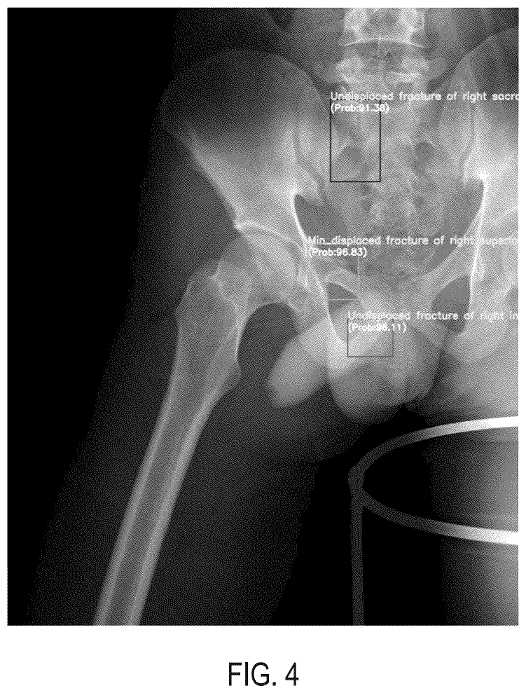

FIGS. 3 and 4 show one or more regions having a main pathology label and a probability level or confidence level associated with each label. As shown in FIGS. 3 and 4, a second deep learning object network may locate one or more fractures in the image and classify each bounding box as a main class label including displaced, undisplaced, displaced_comm, and min_displaced. The system may calculate the IoU between bounding boxes. If the IoU is greater than or equal than 0.5, the system may merge the class labels of two networks. A third classification network may receive all of the bounding boxes and perform sub-attribute classification.

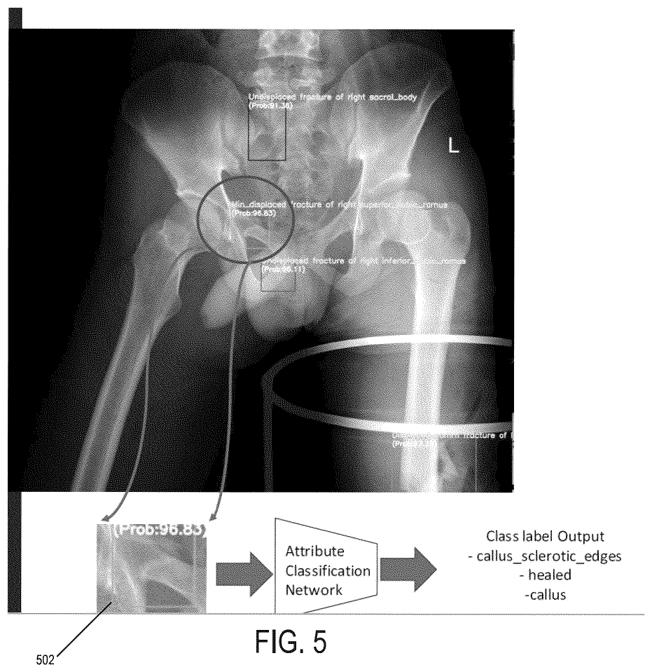

FIG. 5 shows that one of the regions 502 has a probability level of 96.83. After this is determined, the system 100 uses an attribute classification network to provide one or more attributes. The system may crop all bounding box regions and feed it to another classification network for sub-attribute classification. The final output may combine class labels from the one or more classification networks including the first network, the second network, and the third network as undisplaced healed fracture of right superior-pubic-ramus. The first network output is right superior-pubic-ramus, the second network output is undisplaced fracture, and the third network output is healed.

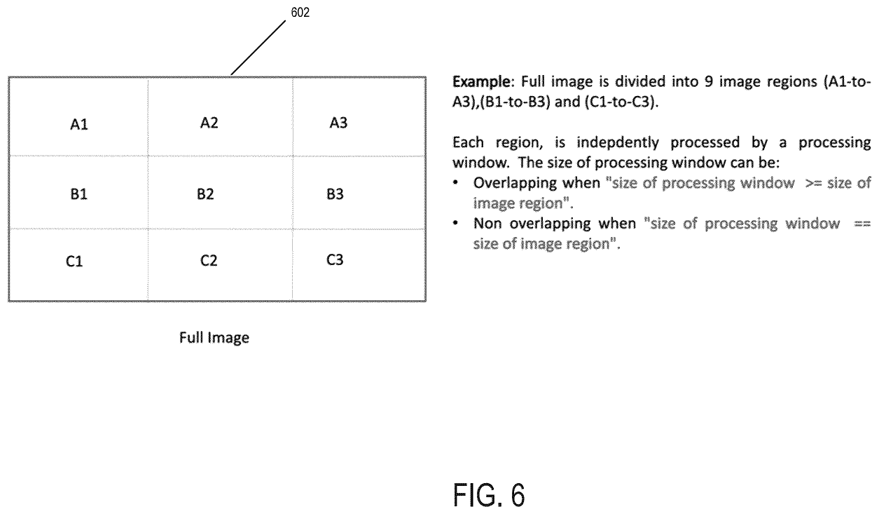

FIG. 6 shows an image 602 divided into regions according to an example of the instant disclosure. As shown in FIG. 6, a full image may be divided into one or more image regions such as A1-A3, B1-B3, and C1-C3. Each region may be independently processed by the system by a processing window. The size of the processing window may be overlapping when a size of the processing window is greater than or equal to a size of an image region. In addition, the size of the processing window may be non-overlapping when a size of the processing window is equal to a size of an image region.

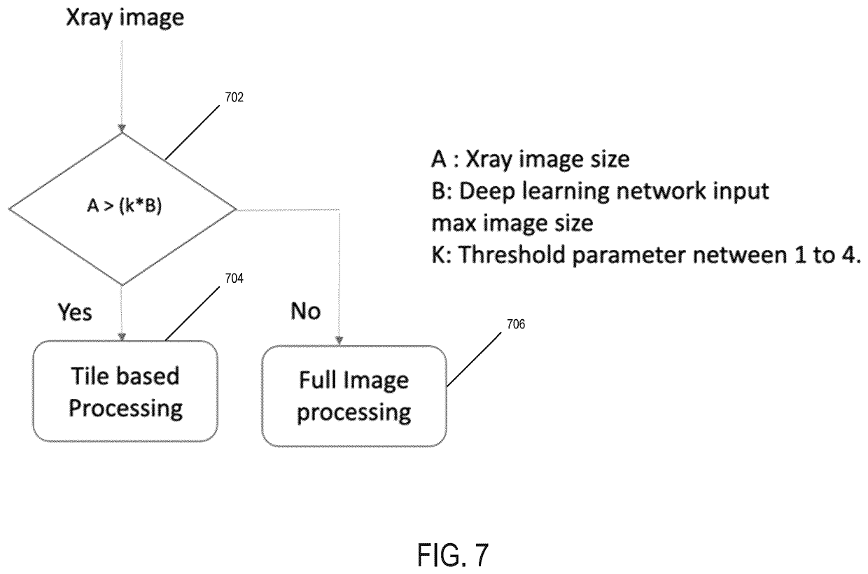

FIG. 7 shows a flow diagram of the diagnostic imaging deep learning system according to an example of the instant disclosure. As shown in FIG. 7, an image such as an X-ray image may have a size equal to A. It is determined whether the image size is greater than a deep learning network input maximum image size multiplied by a threshold parameter of one to four 702. If the image size is greater than the maximum image size, the system may use tile based processing 704 otherwise the system may perform full image processing 706. The image size processing mode may be determined during run time.

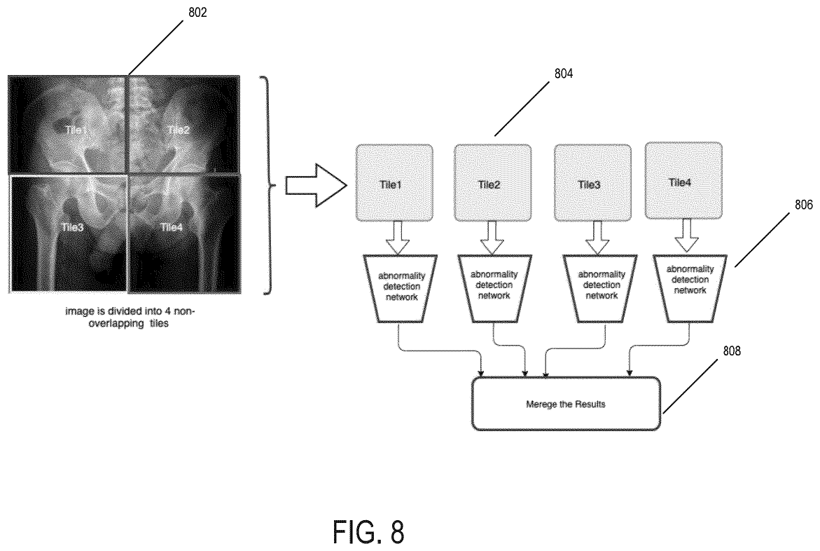

FIGS. 8-10 show an image divided into tiles by the system according to an example of the instant disclosure. As shown in FIG. 8, an image 802 may be divided into four non overlapping tiles 804. Each tile may be processed using an abnormality detection network 806 and the results of each tile may be merged together 808.

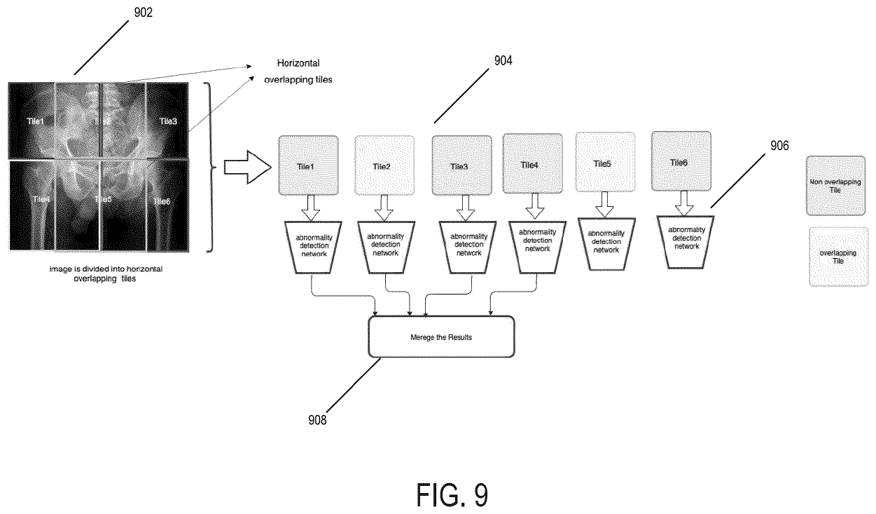

As shown in FIG. 9, an image 902 may be divided into six horizontal overlapping tiles 904. Each tile may be processed using an abnormality detection network 906 and the results of each tile may be merged together 908.

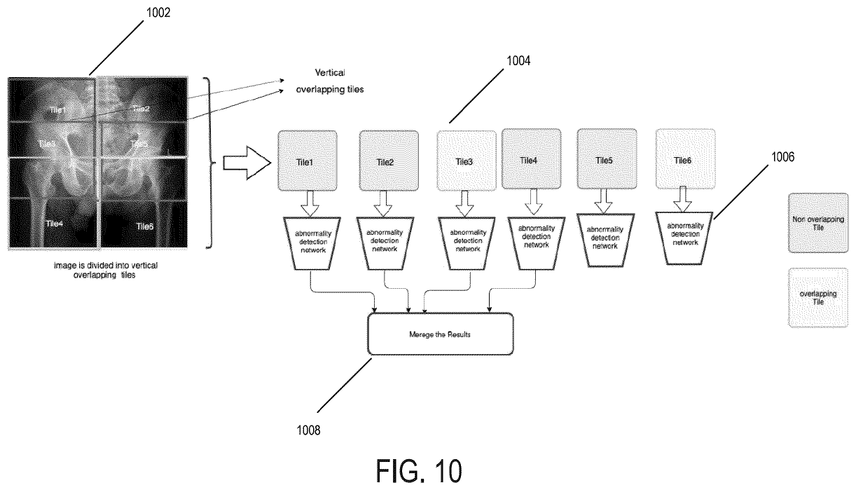

As shown in FIG. 10, an image 1002 may be divided into six vertical overlapping tiles 1004. Each tile may be processed using an abnormality detection network 1006 and the results of each tile may be merged together 1008.

In one example, the image may be preprocessed by an adaptive image resize algorithm. The adaptive image size algorithm has two operational modes including a full image processing mode and a tile based image processing mode.

In the full image processing mode, a full image can be preprocessed by standard preprocessing algorithms such as normalization, zero mean etc., and fed to other processing blocks.

In the tile-based image processing mode, the image can be divided into multiple overlapping or non-overlapping regions, known as a tile. Each tile can be preprocessed by standard preprocessing algorithms and processed by a deep learning network. After all the regions are processed, duplicate bounding boxes are merged/removed using a post processing algorithm such as the non-maximum-suppression algorithm. In tile mode, the image may be processed using non-overlapping mode, horizontal overlapping mode, and/or vertical overlapping mode. The tile-based image processing may retain a high resolution of the image such that during processing details can be retained.

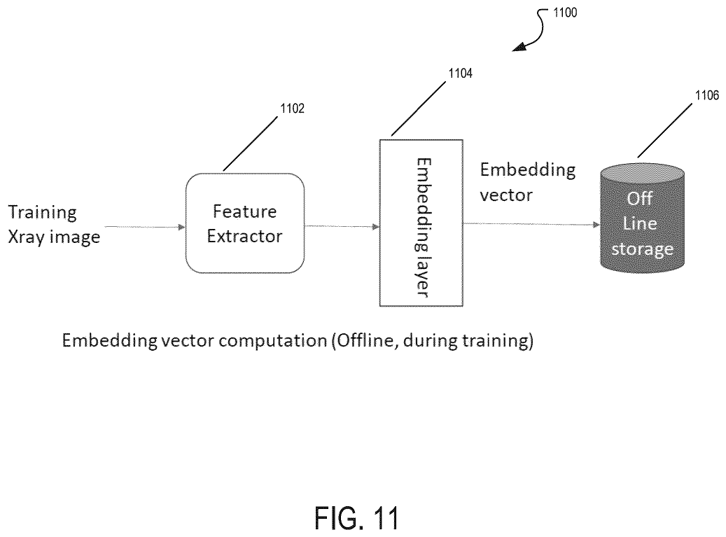

FIG. 11 shows a flow diagram 1100 associated with training according to an example of the instant disclosure. As shown in FIG. 11, the training may include sending the image to a feature extractor 1102 followed by an embedding layer 1104. The image may be stored as an embedding vector in offline storage 1106.

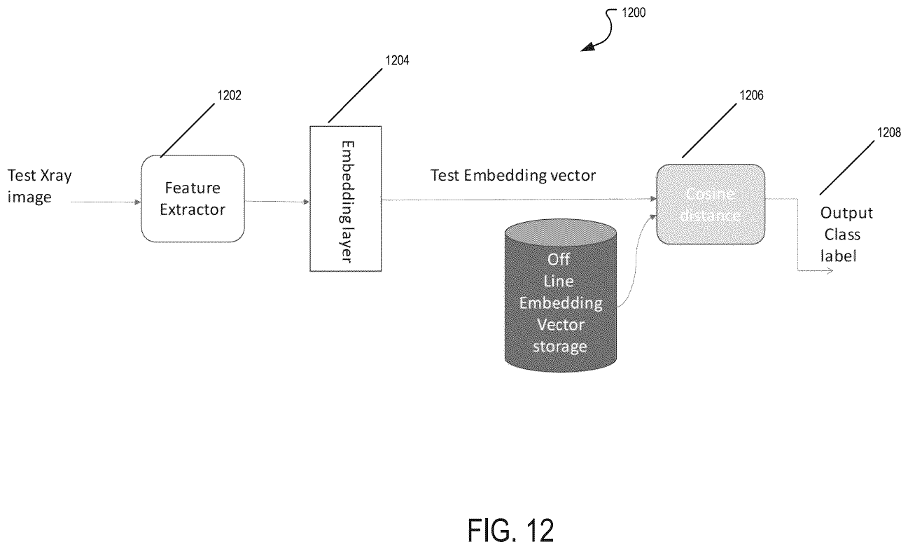

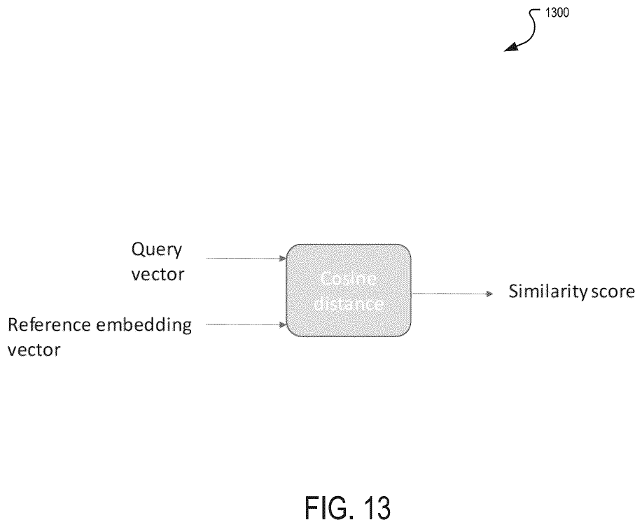

FIGS. 12-16 show flow diagrams associated with processing a diagnostic image according to an example of the instant disclosure. As shown in FIG. 12, the system 100 may perform steps associated with a flow diagram 1200. A test diagnostic image or X-ray image may be input to a feature extractor 1202 followed by an embedding layer 1204. A test embedding vector may be sent as input to a cosine distance determination 1206 along with information from an offline embedding vector storage. An output class label 1208 may be generated by the cosine distance determination. FIG. 13 shows a cosine distance determination 1300. As shown in FIG. 13, the system 100 may input a query vector and a reference embedding vector to a cosine distance determination to determine a similarity score.

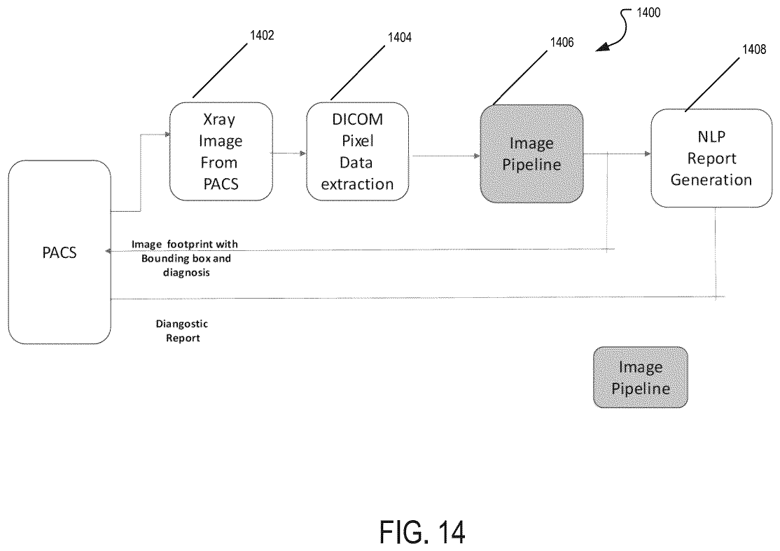

FIG. 14 shows a flow diagram 1400. As shown in FIG. 14, an image may be received from a server 1402, e.g., a picture archiving and communication system (PACS) server. The system may perform pixel data extraction 1404 and extracted pixel array data may be processed by an image pipeline 1406. A diagnostic report may be generated 1408 and the report may be sent to the server.

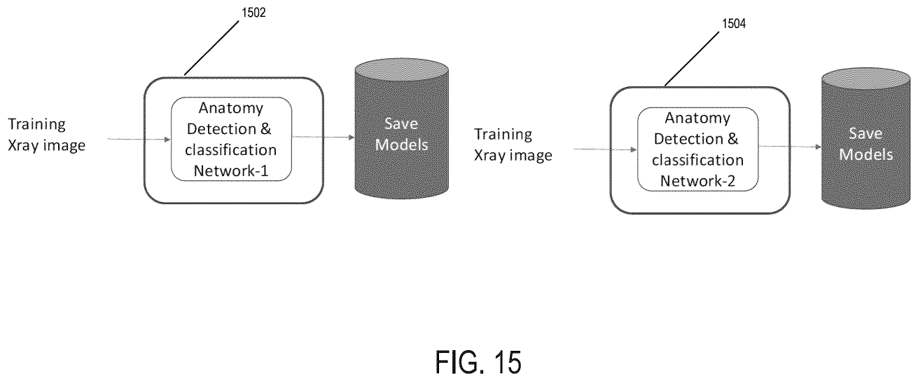

FIG. 15 shows that a training image may be sent to an anatomy detection and classification network one 1502 and saved in a database of models. In addition, FIG. 15 shows that a training image may be sent to an anatomy detection and classification network two 1504 and saved in a database of models.

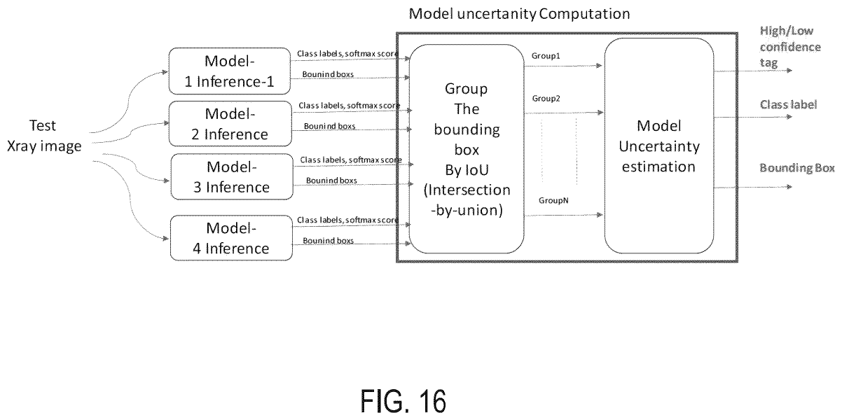

FIG. 16 shows that a test image may be sent to one or more models. In this case, the image may be sent to four models. The system 100 may perform model uncertainty computation by grouping bounding box information by IoU. This may result in a model uncertainty estimation including a high/low confidence tag, a class label, and a bounding box.

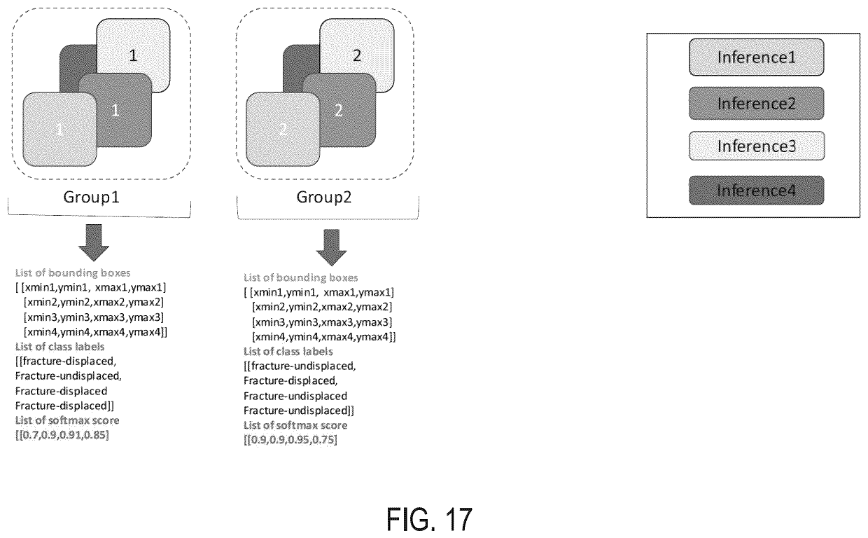

FIG. 17 shows an example of a processed diagnostic image according to an example of the instant disclosure. The image may be processed by a first network and a second network, among others. As shown in FIG. 17, there is a list or array of bounding boxes, a list or array of class labels, and a list or array of a softmax score. The system may generate an output bounding box and a class label for each output. Each output may be a true positive or a false positive. Each output bounding box may be provided with a confidence tag, value, or number. As an example, the confidence tag may provide a prediction certainty or uncertainty. Prediction uncertainty may be derived or based on data uncertainty and/or model uncertainty. Data uncertainty may come into play when input data is out of training distribution. Model uncertainty may be related to training multiple models. During inference time, image inference may be based on multiple models. The results from inference models may be grouped based on IoU of the bounding boxes. For each group of bounding boxes, a model uncertainty may be determined.

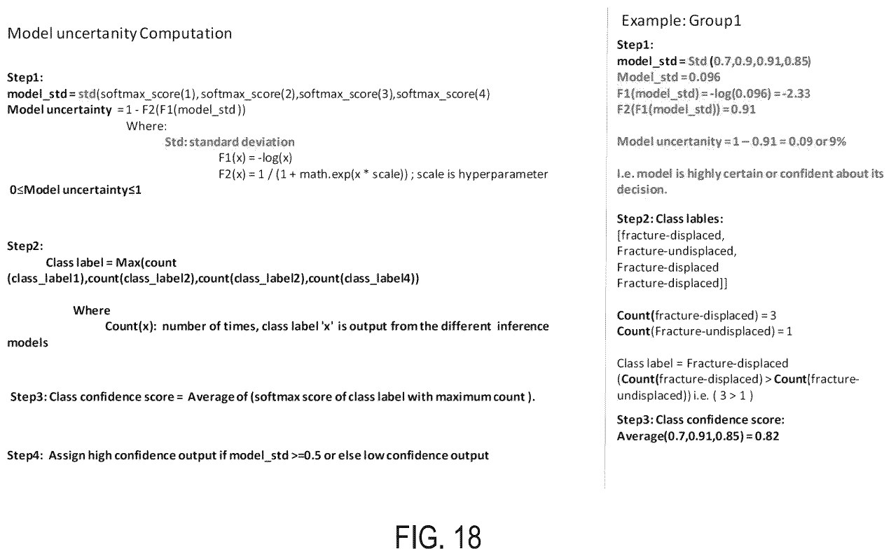

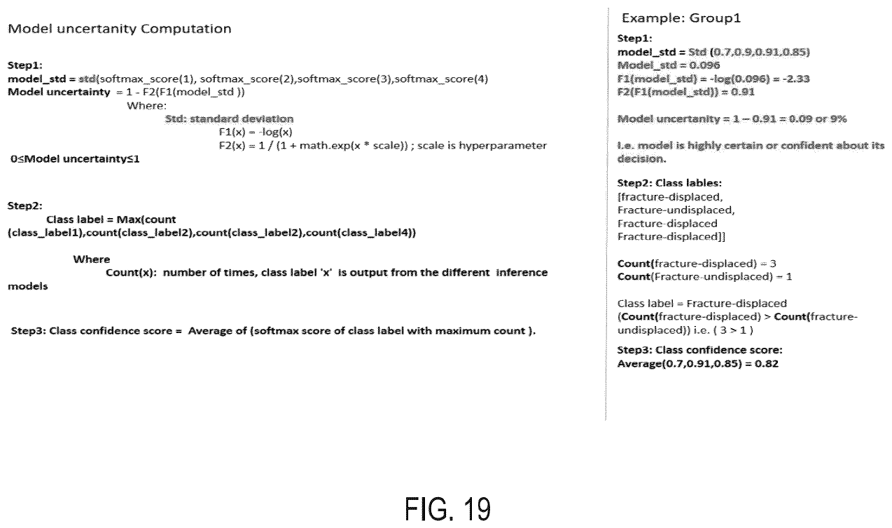

FIGS. 18-19 show model uncertainty computation according to an example of the instant disclosure.

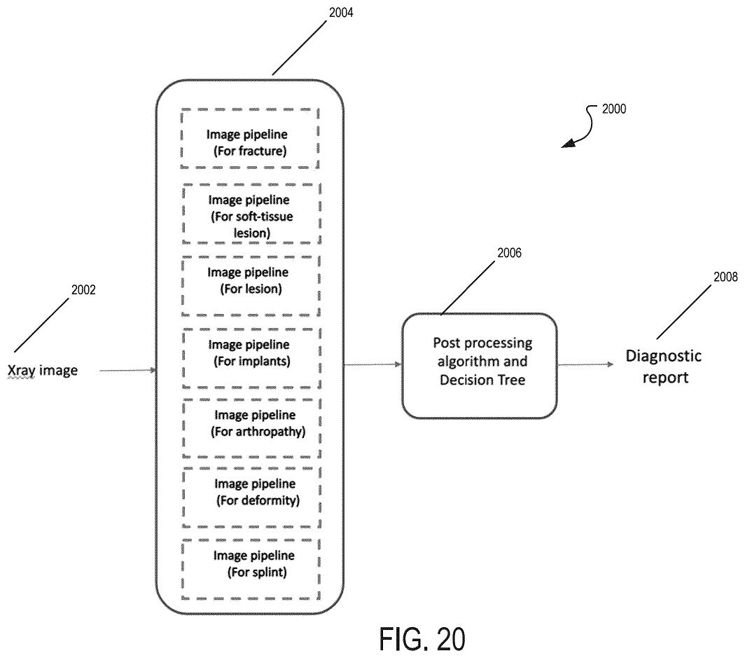

FIG. 20 shows a flow diagram 2000 associated with an image pipeline according to an example of the instant disclosure. As shown in FIG. 20, the system can combine output from multiple deep learning networks. Each network can locate and classify one or more pathology class. Each image may have one or more pathologies or abnormalities including fractures, soft tissue lesions, lesions, deformities, arthropathy, implants, etc. FIG. 20 shows multiple deep learning networks that can be trained to detect and classify abnormalities independently. The output of the one or more networks can be processed by the post processing algorithm. The post processing algorithm can analyze one or more output bounding boxes and labels and remove any false positives. The system may provide a decision tree such as a hierarchical tree having a plurality of nodes to traverse. Each node may be associated with one diagnostic outcome. The input to the decision tree may include the one or more bounding boxes, one or more abnormality class labels, and one or more confidence levels. The output may be a natural language processing (NLP) generated report.

A diagnostic image 2002 such as an X-ray image may be input into one or more image pipelines 2004 such as an image pipeline for fracture, an image pipeline for soft-tissue lesion, an image pipeline for lesion, an image pipeline for implants, an image pipeline for arthropathy, an image pipeline for deformity, and an image pipeline for split. The output from the one or more image pipelines 2004 may be provided to the post processing algorithm 2006 and decision tree to produce a diagnostic report 2008.

The system 100 may generate pathology signatures/descriptors on the fly and compare the signatures/descriptors with stored signatures/descriptors to determine a class label of pathologies/abnormalities in a test image. A region or subsection of each image may be cropped and fed as input to a multi-label classifier. The multi-label classifier may preprocess the cropped region having a fixed image size of X*Y. The multi-label classifier may compute a signature/descriptor of the fixed image. The signature/descriptor may be a vector representation of the fixed image. The multi-label classifier may compare the computed signature/descriptor to offline computed signatures/descriptors having multiple pathology class labels and find a closest match.

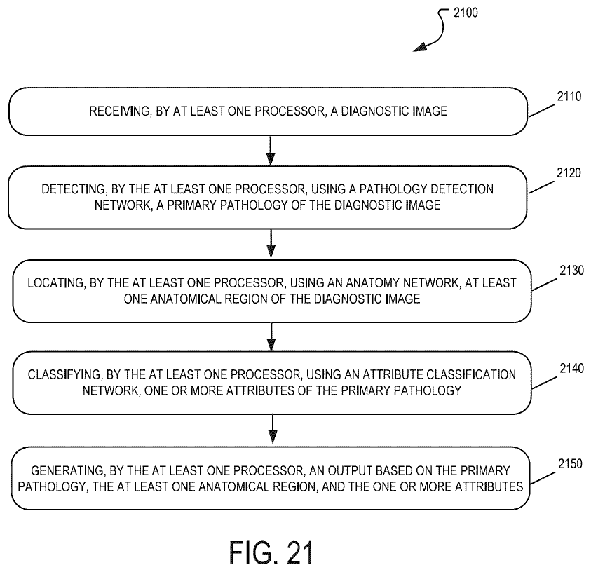

FIG. 21 illustrates an example method 2100 for processing a diagnostic image. Although the example method 2100 depicts a particular sequence of operations, the sequence may be altered without departing from the scope of the present disclosure. For example, some of the operations depicted may be performed in parallel or in a different sequence that does not materially affect the function of the method 2100. In other examples, different components of an example device or system that implements the method 2100 may perform functions at substantially the same time or in a specific sequence.

According to some examples, the method 2100 includes receiving, by at least one process, a diagnostic image at block 2110. The diagnostic image may be an X-ray image or a different type of image. According to some examples, the method 2100 includes detecting, by the at least one processor, using a pathology detection network, a primary pathology of the diagnostic image at block 2120.

According to some examples, the method 2100 includes locating, by the at least one processor, using an anatomy network, at least one anatomical region of the diagnostic image at block 2130.

According to some examples, the method 2100 includes classifying, by the at least one processor, using an attribute classification network, one or more attributes of the primary pathology at block 2140.

According to some examples, the method 2100 includes generating, by the at least one processor, an output based on the primary pathology, the at least one anatomical region, and the one or more attributes at block 2150.

According to some examples, the method 2100 may include determining, for each detected primary pathology, whether a location of the primary pathology overlaps with at least one of the located anatomical regions. In addition, the classifying may include, for each detected primary pathology, discarding the primary pathology if the location of the primary pathology does not overlap with at least one of the located anatomical regions. In addition, the classifying may further include, if the locations of a plurality of the primary pathologies are co-located with one of the at least one located anatomical regions, determining a priority for each of the co-located primary pathologies and discarding at least one of the co-located primary pathologies based on the determined priorities.

According to some examples, the method 2100 may include cropping the diagnostic image to generate at least one classification image based on pathology and anatomical region. The output may be the at least one classification image. In addition, the at least one classification image may have a fixed image size.

According to some examples, the method 2100 may include calculating a vector representation for the at least one classification image, comparing the vector representation with one or more predetermined vector representations, and assigning a classification label to the classification image based on a result of the comparison.

According to some examples, the method 2100 may include generating a natural language processing (NLP) report based on the primary pathology, the anatomical region, and the attributes.

According to some examples, the method 2100 may include pre-processing the diagnostic image.

In addition, the pre-processing may include dividing the diagnostic images into a plurality of image tiles. The method 2100 may further include comparing detected primary pathologies and anatomical regions from the plurality of tiles and discarding duplicate primary pathologies and anatomical regions based on the comparison.

The method 2100 may include image pre-processing of a diagnostic image such as an X-ray image. In one example, the image may be divided into one or more tiles and the one or more tiles may be processed. The intermediate results may be stored in a buffer during processing. In another example, the image may be processed as a full image. The method 2100 may include use of one or more pathology detection networks to find one or more pathologies in the image. After the one or more tiles are processed or the full image is processed, the method 2100 may include post processing. After the post processing, the method may include model uncertainty estimation. The method may include finding overlapping bounding boxes with anatomy detection networks. The method may include cropping one or more regions in the image having an abnormality detected. Next, the method may include attribute classification networks. Next, the method may include use of a decision tree and NLP diagnostic report generation. The diagnostic report may be sent to the server.

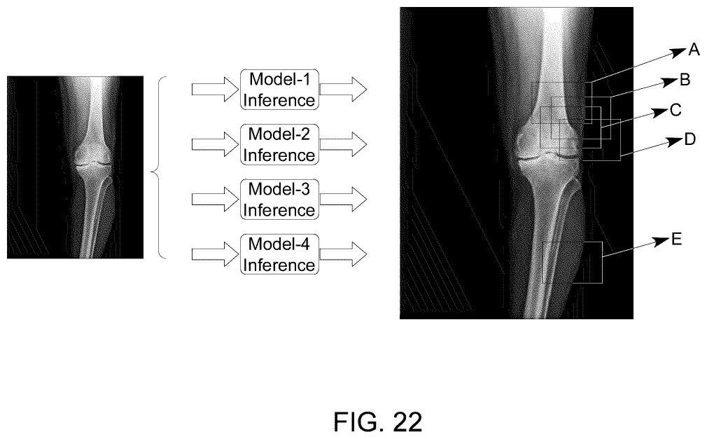

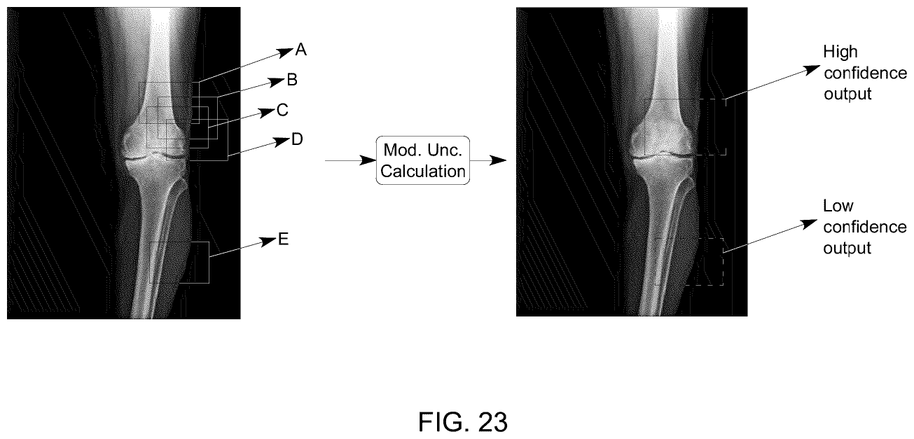

Referring to FIGS. 22 and 23, examples showing high and low confidence output on an output x-ray image are shown. In the example in FIGS. 22 and 23, there are four object detection models, processing a knee-AP x-ray. The output of the model-1 is shown in bounding box A, the output of the model-2 is shown in bounding box B, the output of the model-3 is shown in bounding box C and the output of the model-4 is shown in the bounding box D.

Model-1 produces two bounding boxes (A and E), one with probability score: 0.7, the second with probability score: 0.8. Model-2 produces one bounding box (B), with probability score: 0.9. Model-3 produces one bounding box (C), with probability score: 0.91. Model-4 produces one bounding box (D), with probability score: 0.85. In the example, the “model uncertainty estimation” algorithm analyzes the five identified bounding boxes produced by the four object detection models and marks them as low and high confidence. It can be seen that even though bounding box E has a high probability score of 0.8, it is marked as a “low confidence”. The bounding boxes A, B, C and D overlap with each other (has more than 0.5 IOU (intersection-of-union)), these bounding boxes are merged to one and marked as a “high confidence”, as shown in FIG. 23.

Conventional methods can learn the embedding of an x-ray image. Methods according to embodiments of the present invention can learn the embedding from anatomical region of the image (see box 110 in FIG. 1). The anatomical region has been detected with one or more conditions or pathologies (see box 104, 106 and 108 in FIG. 1).

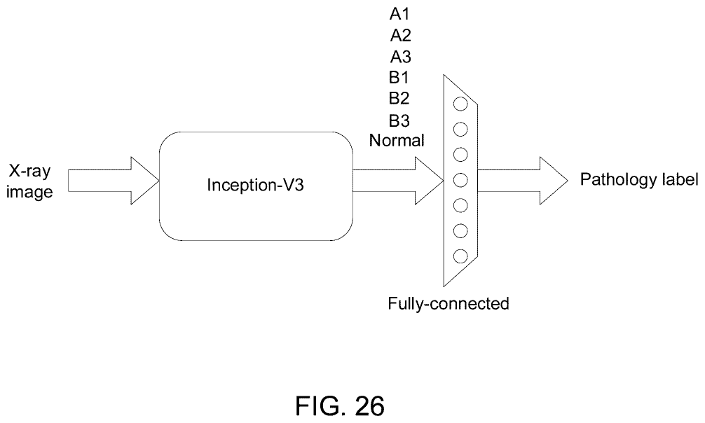

Conventional methods can learn the fracture and its subclassification using a SoftMax layer. The learning process jointly learns the fracture main classification label and its subclassification label (see FIG. 26). Conventional methods do not classify the fracture and its subclassification in hierarchical manner, rather such methods treats fracture and its subclassification as a competition class in the SoftMax layer.

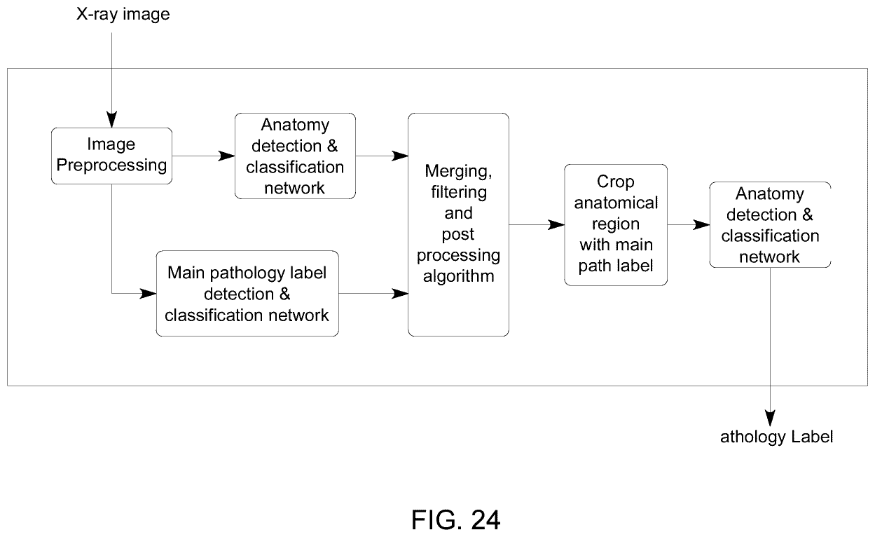

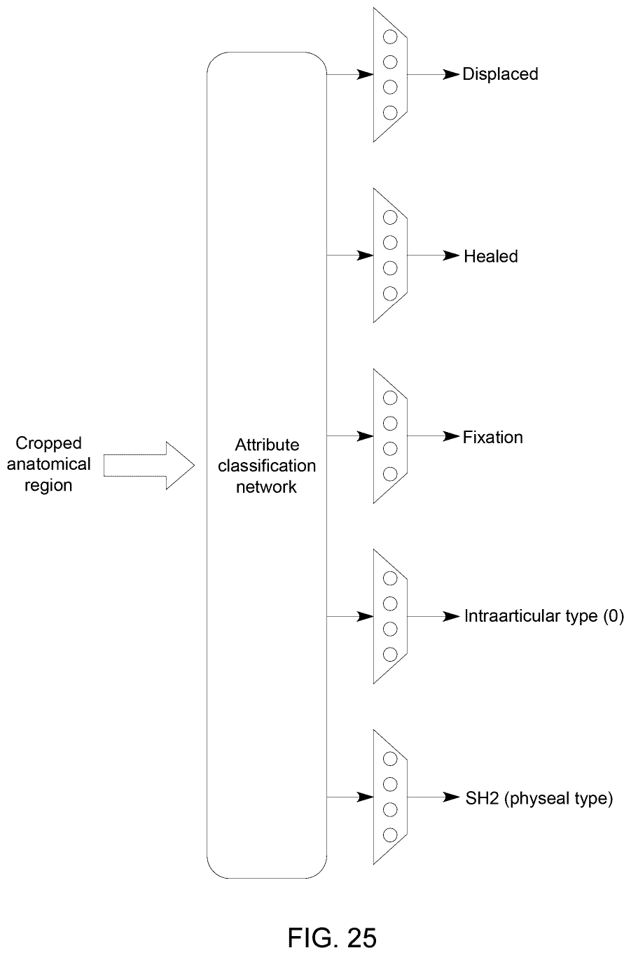

Aspects of the present invention, however, can learn the main class and its sub-classification as follows, referring to FIG. 1 and FIG. 24. Methods, according to aspects of the present invention can first learn the main pathology class, for example, fracture at C2 in FIG. 24. Then the method can learn the anatomical region which has fracture, for example “right proximal femur” as shown at C1 in FIG. 24. Then, the method can crop the anatomical region from x-ray image, for example “right proximal femur” at C3 and C4 in FIG. 24. Then, the attribute classification network can perform subclassification using multi-label classification as shown at C5 in FIG. 24. Multi-label classification can classify multiple sub classification in parallel as illustrated in FIG. 25.

Conventional methods use embedding in context of training the deep learning classifier. The embedding vectors are used for the keyword from diagnostic reports, and then used to train the classifier. Aspects of the present invention, as shown in FIGS. 11 through 13, can learn the embedding vector for one or more conditions or pathologies during training. The learned embeddings are stored and used for inference using query vector (see FIG. 13), to learn the similarity score.

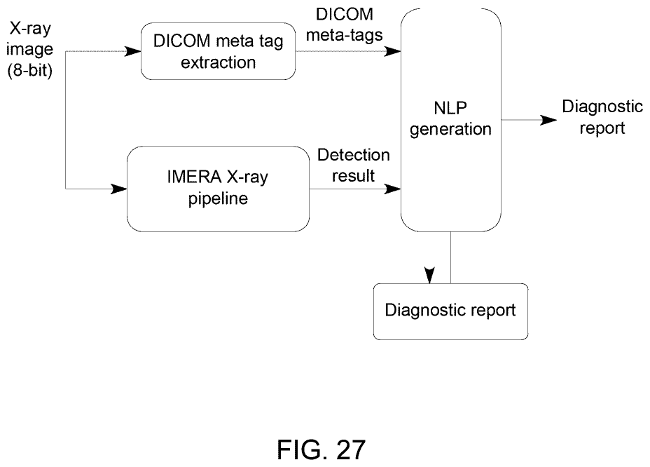

Conventional methods propose detected conditions or pathologies to be a static overlay over the X-ray image. The overlay may indicate to the practitioner which regions of the x-ray contain which detected conditions or pathologies. Methods according to aspects of the present invention, referring to FIGS. 20 and 27, can generate diagnostic reports where the practitioner gets a detailed diagnostic report with information about the detected conditions or pathologies, therefore eliminating the need of manually writing the diagnostic report.

FIG. 27 illustrates an end-to-end block diagram to generate the diagnostic report. The dicom image metatags can include, as examples, patient name, patient age, manufacture name, practitioner name, body view, body part, date and the like. The iMERA x-ray pipeline can provide detection results to the diagnostic report, such as, for a fracture, the displacement type, character type, associative type, intraarticular type, physeal type, fracture body part, and the like. The detection results can also include details about arthritis, dislocations, lesions, soft tissue lesions, implants and the like.

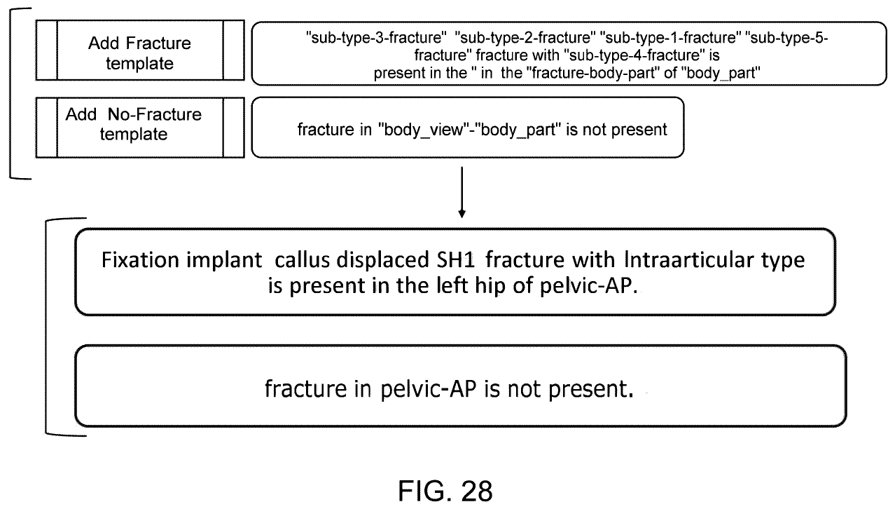

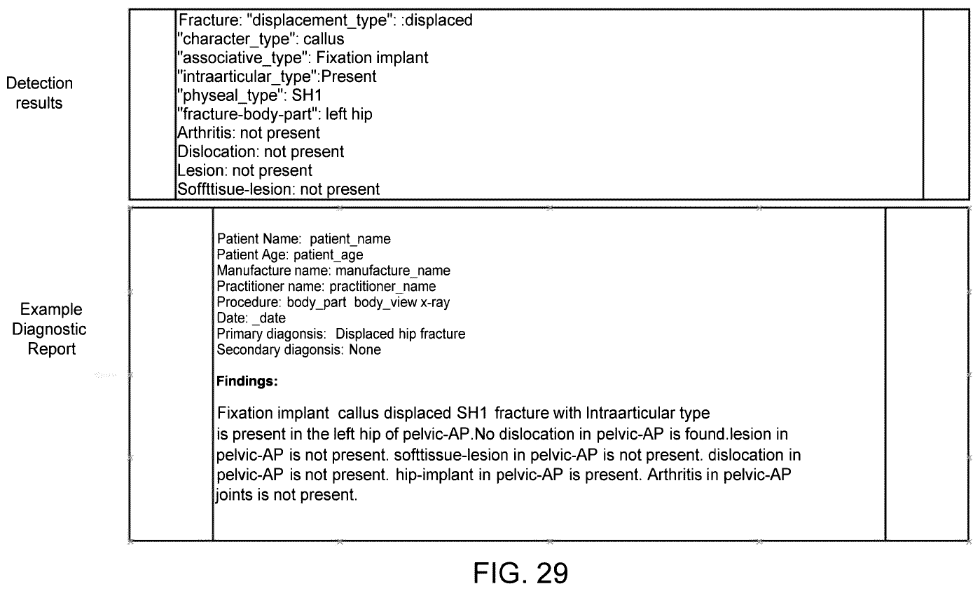

A decision tree can be used in the “NLP generation” block of FIG. 27. Depending upon the presence of abnormality, the NLP generation block decides to use one or more templates, such as a template for each of the detection results. FIG. 28 shows the two templates, one for the presence of fracture and a second for the absence of fracture. FIG. 29 shows the iMERA AI detection results and a corresponding example diagnostic report.

In some embodiments, aspects of the present invention can provide a method for segmentation guided process for analyzing x-ray images to locate and classify single or multiple abnormalities main class labels (for example, fracture, lesions, soft tissue, arthritis or the like) and their hierarchical subclassification class labels.

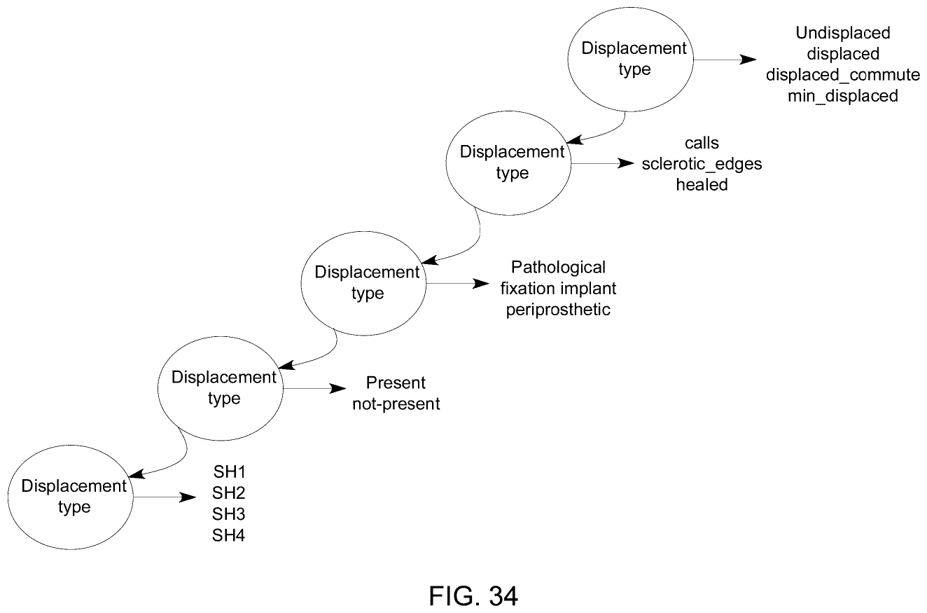

Here, a main class label is defined as a primary class label while hierarchical subclassification class labels are defined as a secondary class labels, derived from the primary (main) class label and having hierarchical dependency. For example, FIG. 34 shows as an example, the hierarchical representation of a fracture where the feature vector of class label lower in hierarchy is derived from the feature vector of class label higher in hierarchy.

A software system for providing methods of the present invention can analyze x-ray image to locate and classify the abnormal findings, if any. There are numerous approaches to analyze x-ray images. For example, one common approach is to use multiple classification and detection deep learning models working in parallel, to locate and classify the abnormal findings. In another approach, single detection and classification deep learning model is used to locate and classify the abnormal findings.

In the method according to an aspect of the present invention, a segmentation guided process can be used for analyzing x-ray images to find multiple abnormalities with hierarchical subclassification class labels.

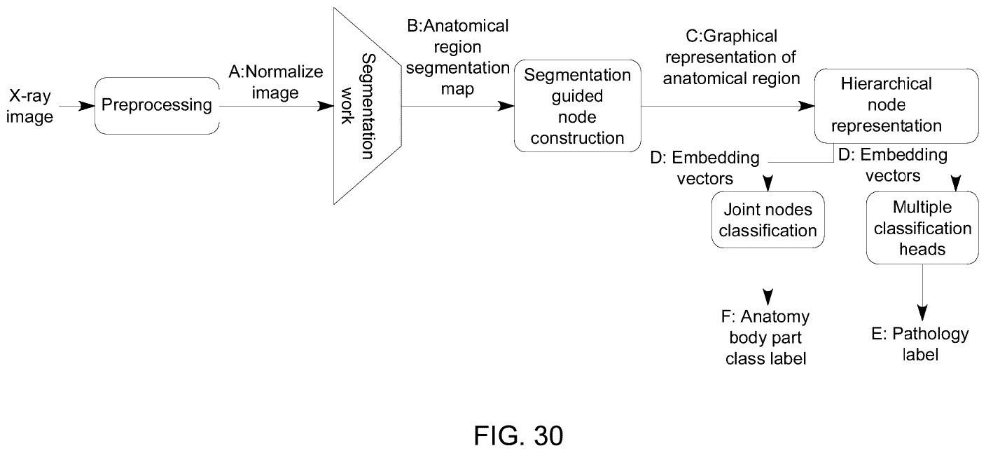

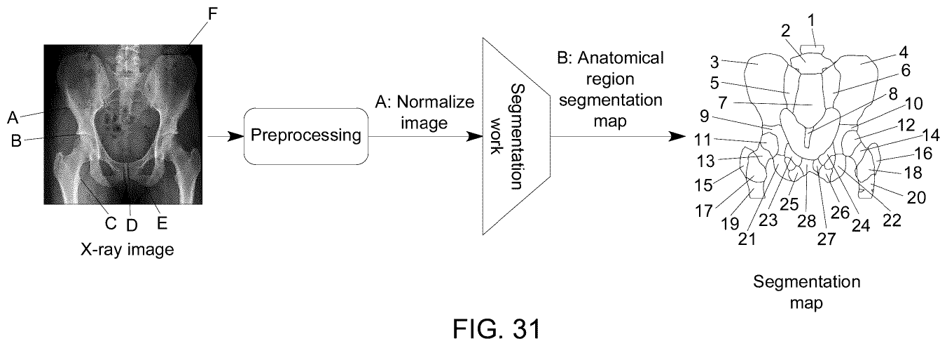

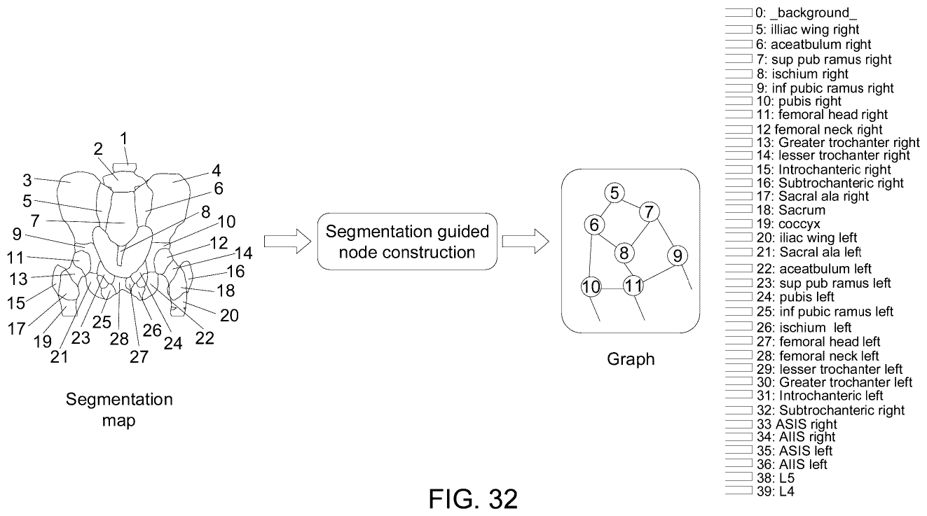

Referring to FIG. 30, an X-ray image is preprocessed using a normalization method. Any standard normalization algorithm is applied to covert image pixels between one of the following ranges: [−1,1], where the following mathematical formula is applied: (img/127.5-1); or [0,1], where the following mathematical formula is applied: (img/255.0). The normalized image is fed to the segmentation network. Any standard deep learning network for segmentation can be used for segmentation. The segmentation network can output a segmentation map. As an example, FIG. 31 illustrates the segmentation map for pelvic-AP x-ray image. FIG. 32 illustrates the color-coding map (shown with numerical references in the Figure, where each numerical reference can be shown as a different color) to represent the color code to anatomical region of the pelvic-AP x-ray image. Pelvic-AP x-ray image is segmented to 39 anatomical region, including background.

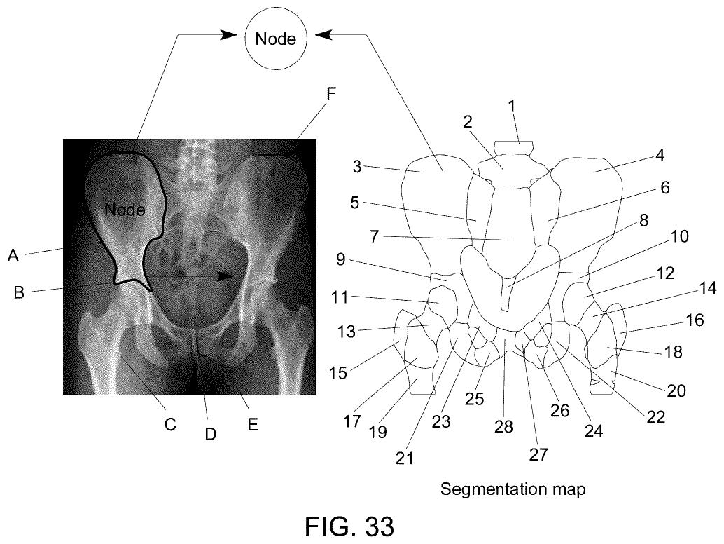

The segmentation map is processed and mapped to a graph that includes nodes and edges. Each node represents an anatomical region of x-ray image. Edge represents the relationship between nodes. The segmentation map is converted to node representation using following steps. (1) The total number of nodes in the graph is equal to the number of color codes in the segmentation map. In the example of pelvic-AP x-ray image, there are 39 color codes, so correspondingly there are 39 nodes. (2) In the segmentation map, starting from top-left to bottom-right, assign all the pixel with same color coding to a single node. (3) Repeat the above step, for all the pixels in the segmentation map. (4) Next, for a given node and its segmentation map, extract the pixels from the x-ray image. This can be done using one-to-one mapping between the location of pixels in the segmentation map of a given node and x-ray image, as shown in FIG. 33. At the end, each unique color code in the segmentation map can represent one node.

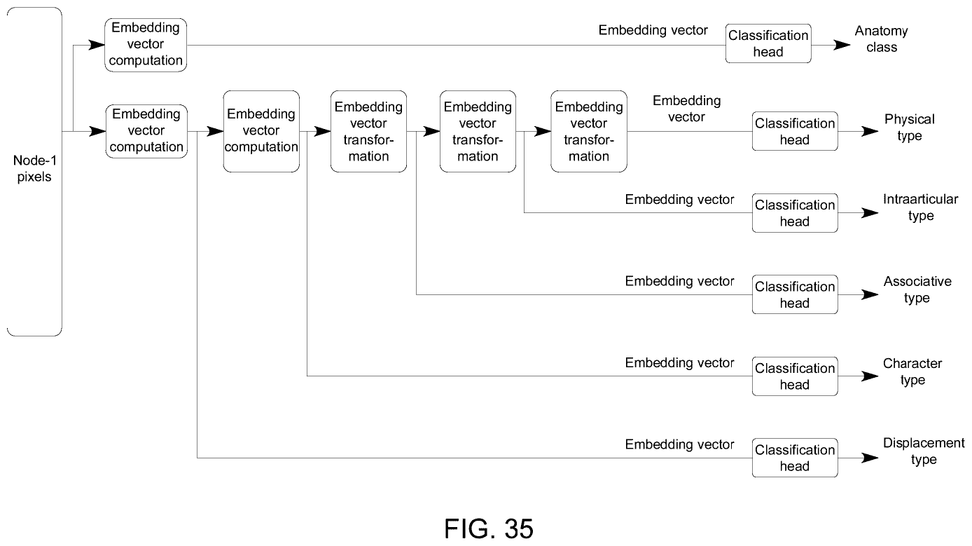

Next, each node in the graph can be processed. Referring to FIG. 35, for each node, its pixels can be taken and processed through a hierarchical embedding vector computation. “Fracture” can be taken as an example. FIG. 34 shows the hierarchical representation of “fracture” class labels. “Displacement type” is in the top in the hierarchy. Any algorithm or deep learning network must first decide if an image has “fracture” and if there exists a “fracture”, what is the “Displacement type”. Then, the algorithm or deep learning network must decide the “Character type” of the “fracture”. It is important to note that the “Character type” of the “fracture” is sub-feature of the main feature “Displacement type”. Therefore, the proposed approach derives the feature of the “Character type” sub classification label from main class label “Displacement type”.

$$Embedding vector for displacement type , {EV}^{displacement} ∈ {R}^{( 1 \times D )}$$

$$Embedding vector for character type , {EV}^{c h a racter} ∈ {R}^{( 1 \times D )}$$

$$Embedding vector for associative type , {EV}^{a s s o ciative} ∈ {R}^{( 1 \times D )}$$

$$Embedding vector for intraarticular type , {EV}^{intraarticular} ∈ {R}^{( 1 \times D )}$$

$$Embedding vector for physeal type , {EV}^{p h y s e a l} ∈ {R}^{( 1 \times D )}$$

$$where D is the embedding vector length$$

$$E {V}^{displacement} = {F}_{displacement} ( NodePixel s ( i , j ) ) , i , j ∈ {R}^{1 \times M} , {R}^{1 \times N}$$

$$where M , N are the dismention of pixel map in node$$

$${EV}^{c h a racter} = {F}_{c h a racter} ( {EV}^{displacement} )$$

$${EV}^{a s s o ciative} = {F}_{a s s o ciative} ( {EV}^{character} )$$

$${EV}^{intraarticular} = {F}_{intraarticular} ( {EV}^{associtive} )$$

$${EV}^{p h y s e a l} = {F}_{p h y s e a l} ( {EV}^{intraarticular} )$$

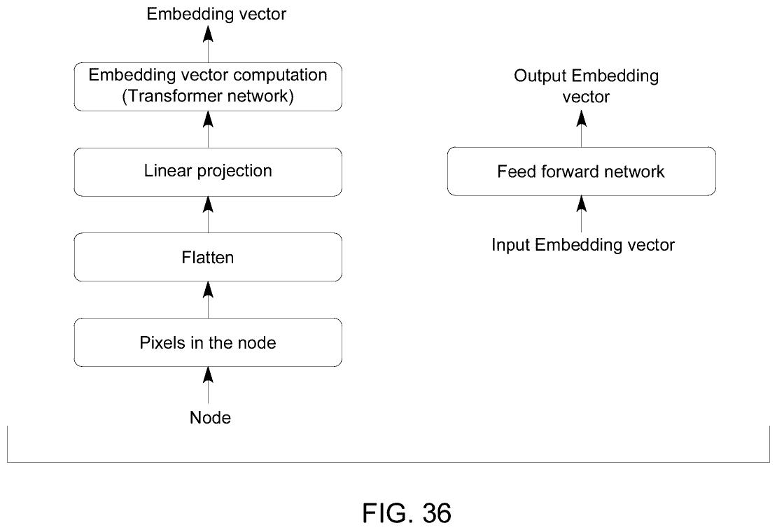

F_{displacement}, F_{character}, F_{associative}, F_{intraarticular} and F_{physeal} are the mapping functions. Mapping functions can be implemented using any standard deep neural network. In FIG. 36, F_{displacement} is implemented using the standard transformer network, to compute the embedding vector, EV_{displacement} Since “displacement” class is the topmost class in hierarchy, its embedding vector is computed using segmentation guided pixel map for the given node in the graph.

The rest of the embedding vectors are derived from their respective classification label, up in the hierarchy. For example, EV_{character} is derived from the EV_{displacement} To derive EV_{character} from the EV_{displacement}, a simple feedforward network can be used. During network training, a simple feedforward network can learn feedforward network weights, to map EV_{displacement} to EV_{character} embedding vectors.

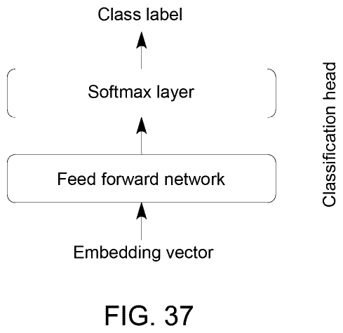

A similar argument is applicable to the rest of sub-classification labels in the hierarchy. FIG. 36 shows the “embedding vector transformation”, a simple feedforward network for such mapping. Finally, learned embedding vectors are fed to classification head in the SoftMax layer as shown in FIG. 37.

The above-mentioned process can be repeated for all the nodes in the graph. This allows network to learn the classification labels and its sub-classification labels for the x-ray image.

The method is not segmentation algorithm or segmentation deep learning model for segmentation x-ray images. The method uses segmentation output to guide the process for classification and detection of single or multiple abnormalities. The method is applied on the segmentation map of the x-ray image. The segmentation map is computed using any of the standard segmentation network, for example DeepLab or seget or similar other deep learning networks.

The method is not a graph convolutional network. In graph convolutional networks, a graph from an image is constructed, without a segmentation map. The method, according to aspects of the present invention, is also not a vision transformer algorithm or vision transformer deep network. Vision transformer divides an image into patches and each patch is encoded for classification using positional embeddings and patch embedding. The following describes how aspects of the present invention differs from graph and vision transformers: (1) The method, according to aspects of the present invention, also constructs a graph from the image, but the graph is constructed using segmentation map. (2) The method, according to aspects of the present invention, also uses embedding for classification and hierarchical subclassification of each node in constructed graph. However, the method does not claim any method for computing embedding. Embedding computation is just another computational block in the process. (3) The method, according to aspects of the present invention, devises applying embedding computation in a hierarchical fashion. The rationale behind this approach is as follows. The merit of such approach is that derived embedding vectors learn features from its ancestor labels, up in the hierarchy. Therefore, one can argue that hierarchical classification labels pose the properties of their ancestors and smoother version of the parent class labels in the hierarchical.

According to another aspect of the present invention, a method is provided for eliminating false bounding boxes appearing in the background region from any standard object detection or classification network applied to x-ray images.

Foreground pixels correspond to the active bone area while background pixels correspond to the remaining non-active bone area. It is noted that background pixels may contain no information (black pixels: pixels with intensity 0 value or near 0 value) or noisy region. It should be noted that this method is applicable to false bounding boxes generated from any object detection network. False bounding boxes decrease the specificity or precision of object detection network, therefore overall decreasing the object detection network performance.

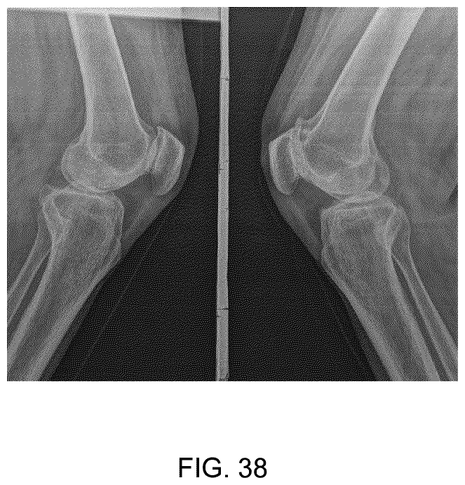

Object detection networks use classifier and localizer networks to classify and draw one or multiple bounding boxes around abnormal area containing pixels with abnormal pattern. When an object detection network analyzes an x-ray image, it analyzes both background pixels and foreground (active) pixels. Background pixels of an image can contain noise. Noise may be defined as presence of an external object which does not constitute the part of foreground (active) pixels. When pixel patterns of noisy pixels are similar to the abnormality (example, fracture, arthritis, bone tumor or the like) pixels, object detection classifier output bounding box, pointing out the presence of abnormality as shown in FIG. 38. Such bounding boxes (appearing in the background region) are false bounding boxes.

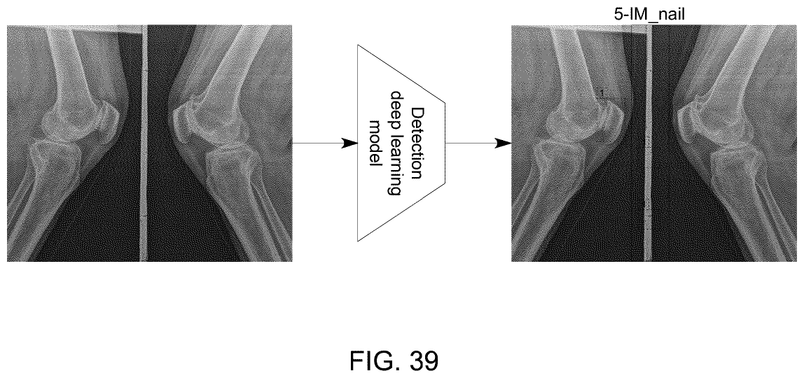

FIGS. 38 through 41 illustrate a knee X-ray image. The knee x-ray image has foreground pixels which form the active knee-bone pixels and background pixels (black pixels and noise in form of external object (metal plate). External object (metal plate) has cracks. When analyzing in isolation, these cracks appear to be a fracture as indicated by the bounding boxes that are not associated with the identified bone in the x-ray. FIG. 39 illustrates object detection deep learning network processing input x-ray image and output x-ray image with bounding boxes. A bounding box indicates the presence of abnormality in the region, enclosed by bounding box coordinates. In FIG. 38, the object detection network draws three bounding boxes, where each of the bounding box is classified as a “fracture”. Out of the three bounding boxes, two bounding boxes are false bounding boxes, as these bounding boxes appear in the background region with noise (external metal plate). The pixel pattern of the false bounding boxes is similar to fracture.

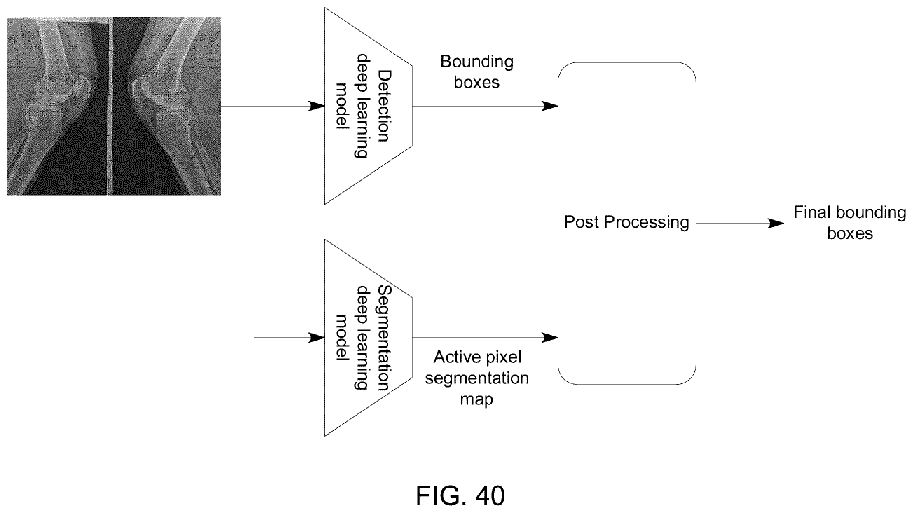

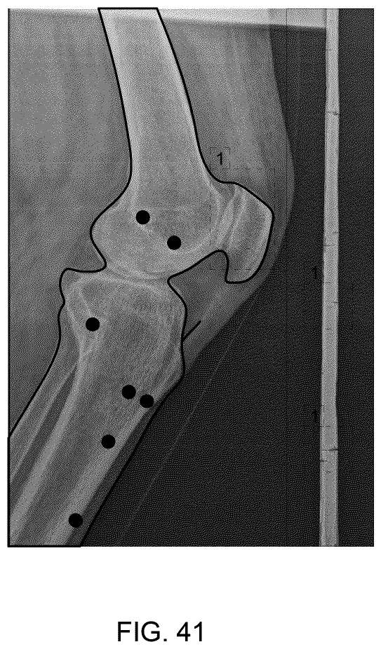

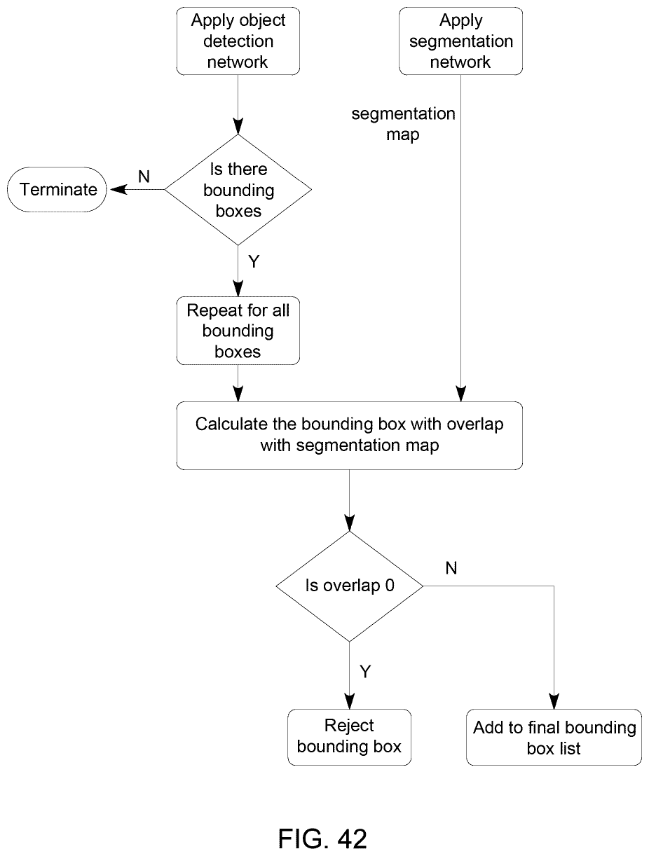

FIG. 40 illustrates a method that includes a segmentation deep learning network and a detection deep learning network. The segmentation deep learning network outputs a segmentation map enclosing only the foreground pixels. FIG. 41 shows the segmentation map by the solid bold black outline. The output of the detection model, if any, and the segmentation map is fed to a post processing block. FIG. 42 shows a post processing algorithm flow-chart. The post processing block checks if a bounding box has any overlap map with the segmentation map. In FIG. 41, it is evident that the bounding boxes appearing in the background noisy region (around external object metal bar) do not overlap with the segmentation map. Therefore, these bounding boxes are eliminated.

Unique aspects of the method according to aspects of the present invention include using the segmentation map to guide the elimination and selection of the bounding boxes. Bounding boxes which do not overlap with the segmentation map are eliminated from the final selection.

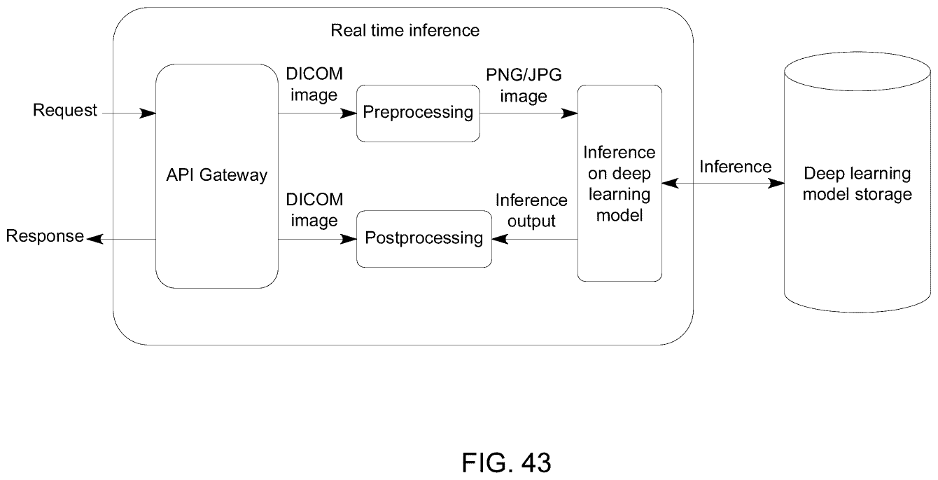

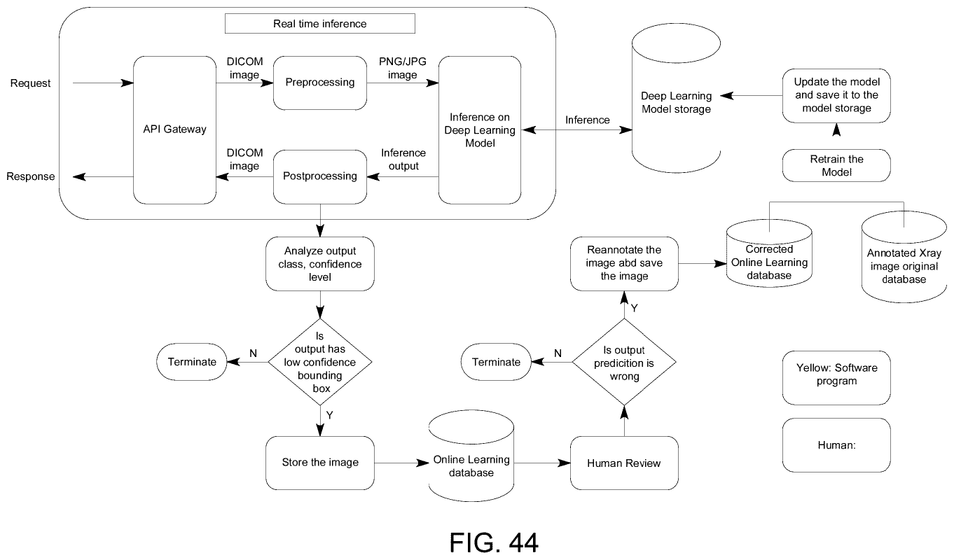

According to another aspect of the present invention, a method is provided for semi-automated online learning based on the low and high confidence level to retrain an existing deep learning model in on-line environment and update it at a regular interval, depending on the output of existing deployed model. FIG. 43 shows a standard deployment of deep learning model in real life. FIG. 44 shows proposed semi-automated online learning method.

As deep learning models need to be updated on a regular basis during real-time inference, the model performance gets worse as the incoming data pattern starts to deviate from the training dataset. Existing approaches involve manually updating the model at regular intervals. In this approach, incoming data and model predictions are collected at regular intervals. Human observers analyze and validate the model predictions. Model false predictions are corrected by humans and the model is retrained on the original training data and newly corrected dataset, to improve the performance. This approach, however, is tedious and requires human innervation to check every single image. To overcome this, a new online learning approach to train the model is described herein.

For the method, according to aspects of the present invention, referring to FIGS. 43 and 44, a software program analyzes the high and low confidence level of deep learning model prediction outputs. If a low confidence level output is found, the software program stores the image to an “online-learning-database” and raises an alarm for human annotator to analyze the image. Low and high confidence levels of deep learning model prediction outputs can be computed from the probability score of the output. For example, a high confidence level is assigned to all of the prediction outputs for which probability score >=0.5. low confidence level is assigned to all of the prediction outputs for which probability score <0.5. Any other method or similar method found in the literature is applicable to make decision about high and low confidence level of the model prediction output. Human annotators can analyze the image and check if model predictions are correct. If no mistake is found, the human annotator discards the image, else if model predictions are wrong, the human annotator annotates the image with correct class labels and bounding boxes. The corrected image is stored in the “corrected online learning database”. Another software program triggers the model training if new images are added to the training dataset and the model is retrained and updated automatically.

The method, according to aspects of the present invention, is a hybrid approach which involves both human and software to regularly update the deep learning model in a real life environment. Since the above method only analyzes the low confidence output, only a subset of the images is needed to be analyzed. This subset is only 20%˜30% of the total image proportions. Therefore, the method, according to aspects of the present invention, significantly reduces the time and computing resources needed for the model update process.

According to another aspect of the present invention, a method is provided for building a training dataset for classification and detection of the abnormal findings in the X-ray dataset. Preparing a training dataset from a large x-ray database is a challenging problem, as an x-ray database may include multiple body-part and body-view x-ray images. However, product requirements can be specific to a particular body-part and body-view x-ray image.

As described above, the methods of the present invention support specific body part and body view x-ray images. To train deep learning model for specific body part and body views, a database of anonymized x-ray images is acquired. However, such database may consist of many bodies parts and views. Manual filtering of the x-ray images of the desired body part and view, from large database, is time consuming process and may take months. To overcome this, a process, according to aspects of the present invention, can include a deep learning model to filter the x-ray images of specific body parts and views.

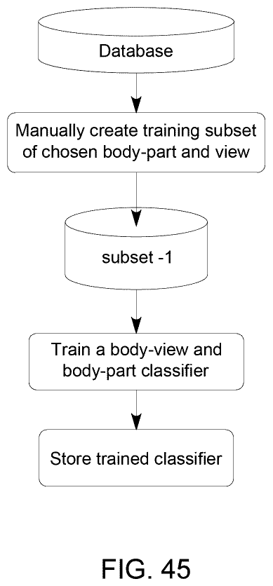

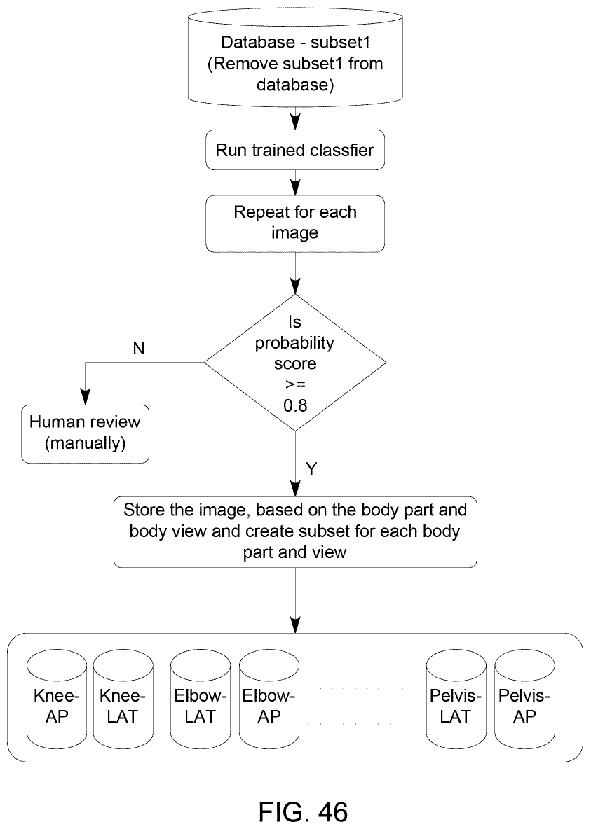

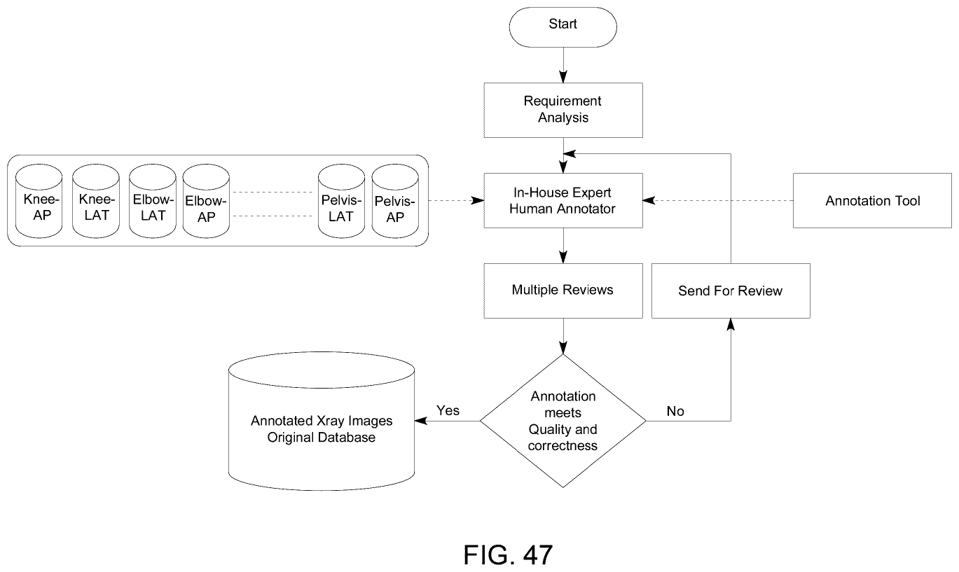

From the large database, a small subset of chosen body-parts and views is filtered. This small subset can be as small as 100 images, for example. A deep learning model classifier is trained on this subset as shown in FIG. 45. For all practical purposes, the deep learning classifier can be any of the deep convolutional network, resent-100, inception-resnet-100 or model classifier such as efficient-net.

Next, as shown in FIG. 44, a trained classifier is run (inference) on the entire database (excluding the subset used for training the classifier). The trained classifier outputs a probability score for each image. If probability score is more than 80%, for example, the image is stored to the appropriate subset, based on the class label. For example, if, for an image, the classifier outputs class label of “Knee-AP”, image is stored to the “Knee-AP” subset. If probability score is less than 80%, the image is sent for human review so that it can be labelled manually.

Next, as shown in FIG. 45, the subset of images with appropriate class labels is annotated by a human annotator. Human annotators draw one or multiple bounding boxes and assign appropriate class label/labels. Annotation is further checked for the quality by multiple reviewers. The annotated X-ray image dataset is trained for detecting single or multiple abnormalities.

There are many conventional approaches, where the methods of the present invention can differ from such conventional approaches by involving human intervention along with the trained classifier to filter the relevant subset of images from the dataset. The method, according to aspects of the present invention, provides a hybrid approach which involves a trained model output (probability score) and human feedback.

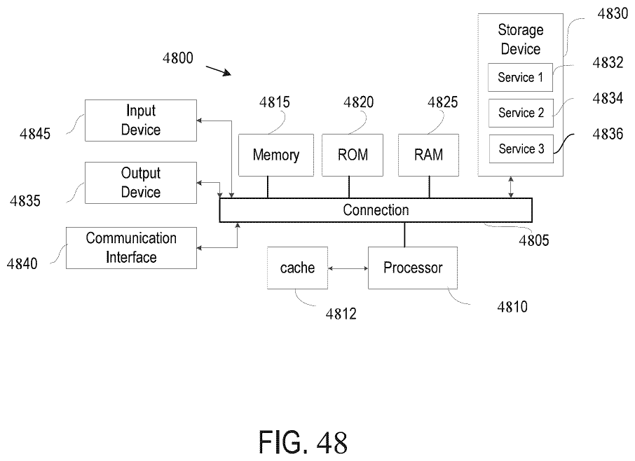

FIG. 48 shows an example of computing system 4800, which can be for example any computing device making up the server, other computing device, or any component thereof in which the components of the system are in communication with each other using connection 4805. Connection 4805 can be a physical connection via a bus, or a direct connection into processor 4810, such as in a chipset architecture. Connection 4805 can also be a virtual connection, networked connection, or logical connection.

In some embodiments, computing system 4800 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 4800 includes at least one processing unit (CPU or processor) 4810 and connection 4805 that couples various system components including system memory 4815, such as read-only memory (ROM) 4820 and random access memory (RAM) 4825 to processor 4810. Computing system 4800 can include a cache of high-speed memory 4812 connected directly with, in close proximity to, or integrated as part of processor 4810.

Processor 4810 can include any general purpose processor and a hardware service or software service, such as services 4832, 4834, and 4836 stored in storage device 4830, configured to control processor 4810 as well as a special-purpose processor where software instructions are incorporated into the actual processor design. Processor 4810 may essentially be a completely self-contained computing system, containing multiple cores or processors, a bus, memory controller, cache, etc. A multi-core processor may be symmetric or asymmetric.

To enable user interaction, computing system 4800 includes an input device 4845, which can represent any number of input mechanisms, such as a microphone for speech, a touch-sensitive screen for gesture or graphical input, keyboard, mouse, motion input, speech, etc. Computing system 4800 can also include output device 4835, which can be one or more of a number of output mechanisms known to those of skill in the art. In some instances, multimodal systems can enable a user to provide multiple types of input/output to communicate with computing system 4800. Computing system 4800 can include communications interface 4840, which can generally govern and manage the user input and system output. There is no restriction on operating on any particular hardware arrangement, and therefore the basic features here may easily be substituted for improved hardware or firmware arrangements as they are developed.

Storage device 4830 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 4830 can include software services, servers, services, etc., that when the code that defines such software is executed by the processor 4810, it causes the system to perform a function. In some embodiments, a hardware service that performs a particular function can include the software component stored in a computer-readable medium in connection with the necessary hardware components, such as processor 4810, connection 4805, output device 4835, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Illustrative examples of the disclosure include:

Aspect 1: A method comprising: receiving, by at least one processor, a diagnostic image, detecting, by the at least one processor using a pathology detection network, a primary pathology of the diagnostic image, locating, by the at least one processor using an anatomy network, at least one anatomical region of the diagnostic image, classifying, by the at least one processor using an attribute classification network, one or more attributes of the primary pathology, and generating, by the at least one processor, an output based on the primary pathology, the at least one anatomical region, and the one or more attributes.

Aspect 2: The method of Aspect 1 wherein the classifying further comprises determining, for each detected primary pathology, whether a location of the primary pathology overlaps with the at least one anatomical region.

Aspect 3: The method of Aspects 1 and 2, wherein the classifying further comprises, for each detected primary pathology, discarding the primary pathology if the location of the primary pathology does not overlap with the at least one anatomical region.

Aspect 4: The method any of Aspects 1 to 3, wherein the classifying further comprises, if more than one location of a plurality of the primary pathologies are co-located with one of the at least one anatomical region, determining a priority for each of the co-located primary pathologies and discarding at least one of the co-located primary pathologies based on the determined priorities.

Aspect 5: The method any of Aspects 1 to 4, further comprising cropping the diagnostic image to generate at least one classification image based on the primary pathology and the at least one anatomical region.

Aspect 6: The method any of Aspects 1 to 5, wherein the output comprises the at least one classification image.

Aspect 7: The method any of Aspects 1 to 6, wherein the at least one classification image has a fixed image size.

Aspect 8: The method of any of Aspects 1 to 7, wherein the classification further comprises: calculating a vector representation for the at least one classification image, comparing the vector representation with one or more predetermined vector representations, and assigning a classification label to the classification image based on a result of the comparison.

Aspect 9: The method of any of Aspects 1 to 8, further comprising generating a natural language processing (NLP) report based on the primary pathology, the anatomical region, and the attributes.

Aspect 10: The method of any of Aspects 1 to 9, further comprising pre-processing the diagnostic image.

Aspect 11: The method of any of Aspects 1 to 10, wherein the pre-processing comprises dividing the diagnostic images into a plurality of image tiles, wherein the classifying comprises comparing detected primary pathologies and anatomical regions from the plurality of tiles and discarding duplicate primary pathologies and anatomical regions based on the comparison.

Aspect 12: The method of any of Aspects 1 to 11, wherein the diagnostic image is an X-ray image.

Aspect 13: A system comprising: at least one processor and a memory having instructions stored thereon and executed by the at least one processor to: receive a diagnostic image, detect using a pathology detection network, a primary pathology of the diagnostic image, locate using an anatomy network, at least one anatomical region of the diagnostic image, classify using an attribute classification network, one or more attributes of the primary pathology, and generate an output based on the primary pathology, the at least one anatomical region, and the one or more attributes.

Aspect 14: A non-transitory computer-readable storage medium, having instructions stored thereon that, when executed by at least one computing device cause the at least one computing device to perform operations, the operations comprising: receiving a diagnostic image, detecting using a pathology detection network, a primary pathology of the diagnostic image, locating using an anatomy network, at least one anatomical region of the diagnostic image, classifying using an attribute classification network, one or more attributes of the primary pathology, and generating an output based on the primary pathology, the at least one anatomical region, and the one or more attributes.

What is claimed is:

1. A method, comprising:
receiving, by at least one processor, a diagnostic image;
pre-processing the diagnostic image by dividing the diagnostic images into a plurality of image tiles;
detecting, by the at least one processor using a pathology detection network, a primary pathology of the diagnostic image;
locating, by the at least one processor using an anatomy network, at least one anatomical region of the diagnostic image;
classifying, by the at least one processor using an attribute classification network, one or more attributes of the primary pathology; and
generating, by the at least one processor, an output based on the primary pathology, the at least one anatomical region, and the one or more attributes.

2. The method of claim 1, wherein the classifying further comprises determining, for each detected primary pathology, whether a location of the primary pathology overlaps with the at least one anatomical region.

3. The method of claim 2, wherein the classifying further comprises, for each detected primary pathology, discarding the primary pathology if the location of the primary pathology does not overlap with the at least one anatomical region.

4. The method of claim 3, wherein the classifying further comprises, if more than one location of a plurality of the primary pathologies are co-located with one of the at least one anatomical region, determining a priority for each of the co-located primary pathologies and discarding at least one of the co-located primary pathologies based on the determined priorities.

5. The method of claim 1, further comprising cropping the diagnostic image to generate at least one classification image based on the primary pathology and the at least one anatomical region.

6. The method of claim 5, wherein the output comprises the at least one classification image.

7. The method of claim 5, wherein the at least one classification image has a fixed image size.

8. The method of claim 7, wherein the classification further comprises:
calculating a vector representation for the at least one classification image;
comparing the vector representation with one or more predetermined vector representations; and
assigning a classification label to the classification image based on a result of the comparison.

9. The method of claim 1, further comprising generating a natural language processing (NLP) report based on the primary pathology, the anatomical region, and the attributes.

10. The method of claim 1, further comprising pre-processing the diagnostic image.

11. The method of claim 10, wherein the pre-processing comprises dividing the diagnostic images into a plurality of image tiles, wherein the classifying comprises comparing detected primary pathologies and anatomical regions from the plurality of tiles and discarding duplicate primary pathologies and anatomical regions based on the comparison.

12. The method of claim 1, wherein the diagnostic image is an X-ray image.

13. The method of claim 1, further comprising generating a segmentation map of the diagnostic image.

14. The method of claim 13, further comprising processing the segmentation map into a graph including nodes and edges, where each node represents an anatomical region of the diagnostic image, and each edge represents a relationship between notes.

15. The method of claim 14, further comprising, for a given node and the segmentation map, extracting pixels from the diagnostic image.

16. The method of claim 15, further comprising determining multiple abnormalities with hierarchical subclassification class labels based on the given node and extracted pixels from the diagnostic image.

17. The method of claim 1, further comprising eliminating false bounding boxes appearing in a background region of the diagnostic image.

18. A system comprising
at least one processor; and
a memory having instructions stored thereon and executed by the at least one processor to:
receive a diagnostic image;
pre-processing the diagnostic image by dividing the diagnostic images into a plurality of image tiles;
detect using a pathology detection network, a primary pathology of the diagnostic image;
locate using an anatomy network, at least one anatomical region of the diagnostic image;
classify using an attribute classification network, one or more attributes of the primary pathology; and
generate an output based on the primary pathology, the at least one anatomical region, and the one or more attributes.

19. A non-transitory computer-readable storage medium, having instructions stored thereon that, when executed by at least one computing device cause the at least one computing device to perform operations, the operations comprising:
receiving a diagnostic image;
pre-processing the diagnostic image by dividing the diagnostic images into a plurality of image tiles;
detecting using a pathology detection network, a primary pathology of the diagnostic image;
locating using an anatomy network, at least one anatomical region of the diagnostic image;
classifying using an attribute classification network, one or more attributes of the primary pathology; and
generating an output based on the primary pathology, the at least one anatomical region, and the one or more attributes.
